(19) United States
(12) Patent Application Publication
(10) Pub. No.: US 2024/0350386 A1
(43) Pub. Date: Oct. 24, 2024

(54) Hair Coloring Compositions Comprising Hydroxyalkyl Amines As Alkalising Agents

(71) Applicant: WELLA GERMANY GMBH, Darmstadt (DE)

(72) Inventors: Viktoriya SENDYUREVA, Darmstadt (DE); Simon GODFREY, Darmstadt (DE); Axel MEYER, Darmstadt (DE); Emad BADALI-BAGHAL, Darmstadt (DE); Teresa BURGAHN, Darmstadt (DE); Bjoern HOFFMANN, Darmstadt (DE); John LANKHOF, Darmstadt (DE)

(21) Appl. No.: 18/682,410

(22) PCT Filed: Aug. 9, 2022
(86) PCT No.: PCT/EP2022/072353

§ 371 Date: Feb. 8, 2024

Related U.S. Application Data
(60) Provisional application No. 63/230,882, filed on Aug. 9, 2021.

(51) Int. Cl. A61K 8/41; A61K 8/22; A61Q 5/10

(52) U.S. Cl. CPC A61K 8/41; A61K 8/22; A61Q 5/10

(57) ABSTRACT

The presently claimed invention relates to oxidative hair colouring compositions comprising at least one alkalizing agent of general formula (I),

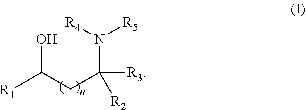

The oxidative hair colouring compositions comprising at least one compound of general formula (I) provide the combined benefit of delivering broader colour shade palette and improved colour intensity with a low level of hair damage, while ensuring low or no odour levels.

### FIELD OF THE INVENTION

The presently claimed invention relates to oxidative hair colouring compositions comprising at least one alkalizing agent of general formula (I),

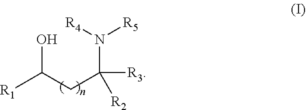

The oxidative hair colouring compositions comprising at least one compound of general formula (I) provide the combined benefit of delivering broader colour shade palette and improved colour intensity with a low level of hair damage, while ensuring low or no odour levels.

### BACKGROUND OF THE INVENTION

Oxidative hair colouring is known since years. Consumers want to be able to colour their hair to many shades, while still having a pleasant and safe usage experience. This requires high lift of hair to deliver a wide range of shades with low or acceptable hair damage while having low or no ammonia smell during the application. In order to deliver a wide range of shades, a large number of dye precursors are used, and they need to be used at sufficient levels to achieve the desired shades.

Ammonia is often used to deliver high lift to obtain a wide variety of dye shades. Delivering high lift causes high damage on hair. Damage of hair includes, e.g., dryness, reduced elasticity, brittleness, split ends, dullness, matte appearance, reduced fullness, rough surface and reduced mechanical strength. Another drawback of the use of ammonia is the heavy smell of ammonia during application.

Monoethanolamine is currently often used within low odour hair colours. It also provides an acceptable level of hair lift and damage. However, it can produce high levels of unwanted dye/alkali adducts which limits the wide range of dye shade formation.

Therefore, there is a need for hair colouring formulations to find an alternative alkali material that provides low odour, while providing low level of formed dye/alkali adducts which enables to form wide range of desired dye shades.

Therefore, there is a need for hair colouring formulations to find an alternative alkali material that provides low odour, and high lift with low or acceptable level of damage, while also giving a low level of formed dye/alkali adducts which enable the formation of a wide range of desired dye shades.

Permanent and demi-permanent oxidative hair colouring products have been used in professional salons and in retail products for use at home for decades. Colouring products typically comprise a tint composition and an oxidizing composition which are packaged separately and mixed immediately before use to form the colouring composition. The tint composition contains so-called oxidative dye precursors which are small molecules (primary dye precursors or coupler dye precursors) and an alkalizing agent, usually ammonia. These primary dyes precursors react with coupler dye precursors in the presence of an oxidizing agent to form larger, coloured dye products within the hair. The oxidizing composition contains the oxidizing agent to enable the colouring process. The oxidizing compositions typically contain a stabilized form of hydrogen peroxide and are sometimes referred to as the developer compositions. The tint composition is thus mixed with the oxidizing composition immediately prior to the application to the hair to be coloured and the resulting mixture of the colouring composition is applied and left on the hair usually for 5 to 50 minutes before rinsing. The mixed colouring composition usually has an alkaline pH between about 8.5 and 10.5.

Oxidative hair colouring compositions comprising ammonia as alkalizing agent within the tint composition are often referred to as permanent hair colourants or “Level 3” hair colourants. Permanent hair colourants are for example marketed under the Koleston Perfect® brand name by Wella Professional in Europe.

Hydrogen peroxide, especially in the presence of ammonia, is capable of bleaching melanin, so that it is possible to obtain shades which are lighter or darker than the natural colour.

Demi-permanent hair colourants, also referred to as “Level 2” colourants, are also typically marketed as two-component systems. They also use primary dye precursors and coupler dye precursors as used in permanent hair colourants but differ in their use of alkalizing agents other than ammonia, in particular monoethanolamine (MEA) or aminomethylpropanol (2-amino-2-methylpropan-1-ol/AMP), and usually function at lower concentrations of hydrogen peroxide, typically 1 to 3 weight % (wt.%) in the mixed colouring composition compared to 3 to 6 or even higher wt. % for permanent “Level 3” colourants. Higher levels of hydrogen peroxide are, however, sometimes used when higher levels of colouring are sought (up to 8 wt. % hydrogen peroxide on the head for some shades). Demi-permanent hair colourants usually cause less melanin bleaching and thus less lift (i.e., removal) of natural hair colour. The resulting dyes may also penetrate less deeply in the hair shaft so that demi-permanent hair colourants can be less long lasting than permanent hair colourants. On the other hand, demi-permanent hair colourants are usually also less damaging to the hair structure than permanent hair colourants and the resulting hair colour may also be more natural looking. A further advantage is that demi-permanent hair colouring compositions do not have the strong ammonia smell of permanent hair colouring compositions and thus have a better consumer experience. A professional brand of demi-permanent hair colourants in Europe is for example Colour Touch® from Wella Professional in Europe. The mixed colouring composition usually has an alkaline pH of between about 8.5 and 10.5.

Demi-permanent hair colouring category also encompasses slightly acidic to low alkali colouring compositions, that do not or only slightly bleach melanin, provide softer colour transformation and deliver none to little hair damage. These mixed compositions usually have pH between 6.5 and 8.5. An example of this type of colouring products is Shinefinity® from Wella Company.

Ammonia-free hair colouring products have been proposed with the goal to provide colouring results close to those obtained with permanent dyes containing ammonia but without an ammonia odour. For example, Schwarzkopf launched in 2009 in Germany a two-component ammonia-free colourant product under the brand name Essensity®. The alkalizing agent used is MEA and a relatively high level of hydrogen peroxide was used, up to 7.7% within the mixed colouring composition.

L'Oréal launched in 2009 in Western Europe a three-component system for professional usage under the brand name INOA®. The INOA® products comprised a fatty composition, a concentrated dye tint composition and an oxidizing composition to be mixed immediately before use. The fatty composition comprises primarily mineral oil and does not comprise an alkalizing agent. The dye tint composition comprises MEA as alkalizing agent. The three components are recommended to be mixed in a 40:16:60 weight ratio. Subsequently L'Oréal evolved the product into a two-component composition again comprising relatively high levels of a fatty component. Several other L'Oréal publications, e.g.: US 2010/0154141 A1, disclose compositions more or less related to the INOA® products. The non-ammonia alkali is preferably chosen from aminomethylpropanol (AMP or 2-amino-2-methyl-1-propanol) and monoethanolamine. None of these disclosures focuses on delivering a wide variety of dye shades which cannot be obtained either with ammonia, MEA or AMP.

U.S. Pat. No. 11,219,578 discloses the challenge of obtaining good colour results in MEA based hair colourants and potential for off-toning versus ammonia-based hair colourants. The solution proposed within this patent is to reduce the level of MEA within such compositions. While this overcomes the challenge of the colour result, by reducing the level of MEA, the amount of lift which can be obtained is also reduced. Therefore, while solving one problem, i.e., the off-tone associated with MEA based hair colourants, another problem is created, i.e., the inability to deliver sufficient hair lift.

Although the prior art discloses using two-or three-component systems to obtain a permanent hair colouring result without ammonia, these component systems are based on the use of aminomethylpropanol (AMP or 2-amino-2-methyl-1-propanol) and monoethanolamine. While both species provide an odour benefit and low damage benefit, they both have significant draw backs such as not delivering the desired colour shade palette.

AMP is not effective at providing the bleaching or lift to the melanin within the hair. This prevents products formulated with AMP to deliver a wide range of shades, as lifting, or bleaching the hair is needed to enable shades which are lighter than the consumer or clients base colour, and to provide higher levels of grey coverage. Thus, AMP is not a good replacement for ammonia to deliver a wide range of shades needed for Level 2 and Level 3 hair colourants.

MEA is able to provide better levels of hair lifting and bleaching and is almost at par with ammonia-based systems. However, MEA leads to significant off-tone of some of the oxidative hair dye species used. Color formulation in the presence of MEA can lead to dye structures with the alkali material incorporated into the colored dye product.

The dye structures with the alkali incorporated are henceforth referred to as dye adducts. Herein after these dye structures will be named as “mono-dye adducts” or “di-dye adducts”. The light absorbing properties of the dye adducts differs from the regular dye species formed in the presence of ammonia, and this is thought to be responsible for the off-tone colour formation observed in the presence of MEA. Differently coloured species are formed concurrently to the desired coloured species. The amount of these dye adducts depends on the choice of primary dye precursor and coupler dye precursor, and the level of MEA and the reaction conditions. A higher level of MEA and a higher pH leads to higher adduct levels.

Thus, formation of mono-dye and/or di-dye can lead to off-tones and prevent the direct reapplication of dye recipes from ammonia colourants into MEA based hair colourants.

There is a need for providing hair colouring compositions that enable the formation of all desired colour shades and intensities and provide no or low levels of lift, while reducing the level of off-toning adduct species of oxidative dyes and giving a low level of hair damage without the formation of any unpleasant odour during use.

### SUMMARY OF THE INVENTION

It is an object of the presently claimed invention to provide hair colouring compositions that enable the formation of all desired colour shades and intensities and provide no or low levels of lift, while reducing the level of off-toning adduct species and giving a low level of hair damage without the formation of any unpleasant odour during use.

The object has been solved by using alkanolamines as an alkalizing agent in an oxidative hair colouring composition.

Accordingly, in one aspect, the presently claimed invention is directed to a hair colouring composition comprising
a) at least one alkalizing agent of the general formula (I),

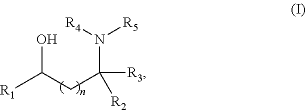

wherein
R1 is hydrogen or linear or branched C1-C6 alkyl which is unsubstituted or substituted with 1, 2, 3 or 4 OH,
R2 is hydrogen or linear or branched C1-C6 alkyl which is unsubstituted or substituted with 1 or 2 OH,
R3 is hydrogen or linear or branched, unsubstituted C2-C6 alkyl,
or
R2 and R3 together with the carbon atoms to which they are bonded form a saturated 5-, 6-, 7-or 8-membered carbocyclic ring that is unsubstituted or substituted with 1, 2, 3, or 4 substituents selected from the group consisting of OH and NH₂,
R4 is hydrogen or linear or branched, unsubstituted C1-C6 alkyl,
R5 is hydrogen or linear or branched, unsubstituted C1-C6 alkyl, and
n is 0, 1, 2 or 3; n
whereby the compound of general formula (I) is not monoethanolamine;
b) at least one oxidizing agent;
c) at least one oxidative primary dye precursor;
d) at least one oxidative coupler dye precursor and
e) at least one buffering agent.

In another aspect, the presently claimed invention is directed to a hair colouring composition which is a two-component hair colouring comprising a tint composition (A) and an oxidizing composition (B); wherein the tint composition (A) comprises
a) at least one alkalizing agent of general formula (I),
c) at least one oxidative primary dye precursor;
d) at least one oxidative coupler dye precursor and
optionally e) at least one buffering agent; and
wherein the oxidizing composition (B) comprises
b) at least one oxidizing agent and
optionally e) at least one buffering agent;.

Another aspect of the presently claimed invention relates to a method for colouring hair by applying the oxidative hair colouring composition as described herein above and below.

Yet another aspect of the presently claimed invention relates to the use of a compound selected from the group consisting of 2-amino-1-propanol, 1-amino-2-propanol, 3-amino-1-propanol, 2-amino-propane-1,3-diol, 1-amino-2-butanol, 4-amino-2-butanol, 4-amino-1-butanol, 3-amino-1-butanol, 3-amino-2-butanol, 2-amino-1-butanol, dimethyl glucamine, 2,5-diaminocyclohexane-1,4-diol, 2-aminocycohexan-1-ol and 2,4,6-triaminocyclohexane-1,3,5-triol as an alkalizing agent for an oxidative hair colouring composition.

### DETAILED DESCRIPTION OF THE INVENTION

The following detailed description is merely exemplary in nature and is not intended to limit the presently claimed invention or the application and uses of the presently claimed invention. Furthermore, there is no intention to be bound by any theory presented in the preceding technical field, background, summary or the following detailed description.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”.

Furthermore, the terms “(a)”, “(b)”, “(c)”, “(d)” etc. and the like in the description and in the claims, are used for distinguishing between similar elements and not necessarily for describing a sequential or chronological order. It is to be understood that the terms so used are interchangeable under appropriate circumstances and that the embodiments of the subject matter described herein are capable of operation in other sequences than described or illustrated herein. In case the terms “(A)”, “(B)” and “(C)” or “(a)”, “(b)”, “(c)”, “(d)”, “(i)”, “(ii)” etc. relate to steps of a method or use or assay there is no time or time interval coherence between the steps, that is, the steps may be carried out simultaneously or there may be time intervals of seconds, minutes, hours, days, weeks, months or even years between such steps, unless otherwise indicated in the application as set forth herein above or below.

The term “about”, when used in relation to a weight ratio or a weight percentage, means that a specific value should be understood as meaning a range of +10%. For example, the weight proportion of a compound of about 1%, means a weight proportion ranging from 0.9 to 1.1%. For example, the weight proportion of a compound of about 2.5%, means a weight proportion ranging from 2.25 to 2.75%.

In the following passages, different aspects of the subject matter are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to “one embodiment” or “preferred embodiment” means that a particular feature, structure or characteristic described in connection with the embodiment is included in at least one embodiment of the presently claimed invention. Thus, appearances of the phrases “in one embodiment” or “in a preferred embodiment” in various places throughout this specification are not necessarily all referring to the same embodiment but may refer to different embodiments of the presently claimed invention. Furthermore, the features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the subject matter, and form different embodiments, as would be understood by those in the art. For example, in the appended claims, any of the claimed embodiments can be used in any combination.

Furthermore, the ranges defined throughout the specification include the end values as well, i.e., a range of 1 to 10 implies that both 1 and 10 are included in the range. For the avoidance of doubt, the applicant shall be entitled to any equivalents according to applicable law.

For the purposes of the presently claimed invention, ‘% by weight’ or ‘wt. %’ as used in the presently claimed invention is with respect to the total weight of the colouring composition. Further, the sum of wt. % of all the compounds, as described herein, in the respective components adds up to 100 wt. %.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

The measurement techniques described hereinabove and herein are well known to a person skilled in the art and therefore do not limit the presently claimed invention.

By “oxidative hair colouring composition”, it is meant a ready-to-use composition which can change the colour of hair on which it is applied, and which comprises an alkalizing agent, an oxidizing agent and oxidative dyes.

By “two-component” oxidative hair colouring composition it is meant an oxidative hair colouring composition which is obtained by mixing shortly before use two compositions: a tint composition and an oxidizing composition. The tint composition comprises the oxidative primary dye precursors, oxidative coupler dye precursors and the alkalizing agent. The oxidizing composition comprises the oxidizing agent.

By “composition” it is meant a composition which is mixed by the user with one or more other composition for preparing the ready-to-use oxidative hair colouring composition to be applied to the hair. Hereinafter, the “hair colouring composition” refers to a use/mixed hair colouring composition.

By “user” it is meant the person preparing the hair colouring composition. The user is for example a professional hair stylist working in a salon and is different from the subject on whose hair the composition is applied, or the user is identical to the person on whose hair the composition is applied. The mixing can take place in a bowl or within a bottle or even through a mixer placed between the stored individual compositions and the point at which it is dispensed to be used. While two component oxidative hair colouring compositions are the most commonly found in the market, there are also others which use three or more components. For example, these colouring compositions comprise a tint composition and a separate alkali composition both of which are mixed in combination with the oxidizing composition to produce the colouring composition. In such a three-component system, the tint composition and the alkali composition are considered to be equivalent to the tint composition of a two-component system.

Alternatively, a tint composition, an anhydrous oxidizer composition and an aqueous composition can be mixed with the anhydrous composition to create the finished mixed colouring composition with the tint composition. In such a three-component system, the anhydrous oxidizer composition and the aqueous composition form the oxidizer composition of the two-component system. An anhydrous dye powder, tablet or granule can also be mixed with an aqueous alkali composition and an oxidizing composition.

Those skilled in the art are aware that there are multiple combinations of the components that can be interchanged to obtain the final mixed hair colouring composition. All these combinations are within the scope of the presently claimed invention which is exemplified in a non-limiting way.

By “lift” (or “lift power”) it is meant the amount of lightening caused by the bleaching of the natural hair pigment melanin by using the colouring composition of the presently claimed invention. The amount of lift provided by different hair colouring compositions can be compared by using a human natural dark hair sample (e.g., dark hair of an individual of Chinese descent) and measuring the change of colour achieved following application of the compositions. The change in colour can be measured using well known parameters such as L*a*b* values. A composition can be said to provide a higher lift than another composition, when the resulting L* value or the so-called dL* value (given by L*_{final}−L*ᵢₙᵢₜᵢₐₗ) measured for a given treated sample of dark hair is higher for that composition than for the other composition, using the same experimental conditions. The denomination Level 2 (herein used interchangeably with “demi-permanent” or “tone-on-tone”) and Level 3 (herein used interchangeably with “permanent”) are commonly used in the hair colour trade to differentiate compositions with medium and high lift.

By “oxidizing agent” it is meant an electron accepting compound suitable for use in hair colouring compositions for removing the natural colour of hair (by destroying the melanin pigment) and reacting with oxidative primary dye precursors to enable the reaction with the oxidative coupler dye precursors to form the colour dye products. The most commonly used oxidizing agent in the art is hydrogen peroxide, however further suitable oxidizing agents that can be used in combination with hydrogen peroxide are described below. Oxidizing agents also include those which produce hydrogen peroxide when contacted with water. For example, sodium percarbonate, sodium perborate and urea peroxide can be used as anhydrous materials, which when contacted with water release hydrogen peroxide. For such oxidizing agents which produce hydrogen peroxide when contacted with water, the wt. % of hydrogen peroxide within mixed colouring composition is considered as the level of b) oxidizing agent.

By “alkalizing agent” it is meant one or more compounds suitable for raising the pH to alkaline level in mixed colouring compositions, in particular to a pH in the range of ≥6.5 to ≤8.5. Generally, the most commonly used alkalizing agents in the art are ammonia and monoethanolamine, however the presently claimed invention involves using an alkalizing agent other than ammonia (herein “non-ammonia, non-monoethanolamine” alkalizing agent), in particular the alkanolamine of general formula (I). The alkali agent may be used to keep the pH of the mixed colouring composition to a pH in the range of ≥6.5 to ≤8.5.

The below materials are being used in the embodiments of the presently claimed invention. The presently claimed invention is described with non-limiting embodiments. The listed ingredients are the preferred features as well as other preferred but non-limiting features of the presently claimed invention.

### Formulation Chassis and Ingredients

Common ingredients for the hair colouring formulations include, but are not limited to: alkalizers, solvents; water, oxidative dyes; direct dyes; oxidizing agents, hydrogen peroxide; radical scavengers; thickeners and/or rheology modifiers; chelants; pH modifiers and buffering agents; carbonate ion sources; peroxymonocarbonate ion sources; anionic, cationic, non-ionic, amphoteric or zwitterionic surfactants, or mixtures thereof; anionic, cationic, non-ionic, amphoteric or zwitterionic polymers, or mixtures thereof; fragrances; enzymes; dispersing agents; peroxide stabilizing agents; antioxidants; catalysts, natural ingredients, e.g. proteins and protein derivatives, and plant extracts; conditioning agents including silicones and cationic polymers, ceramides, preserving agents, pigments, and opacifiers and pearling agents (such as titanium dioxide and mica). Some adjuvants referred to above, but not specifically described below, which are suitable are listed in the International Cosmetics Ingredient Dictionary and Handbook, (8th ed.; The Cosmetics, Toiletry, and Fragrance Association). Particularly, vol. 2, sections 3 (Chemical Classes) and 4 (Functions) are useful in identifying specific adjuvants to achieve a particular purpose or multipurpose.

Examples of formulation chassis for the tint component can be found for example in patent literature or adapted from existing commercial products, especially products comprising non-ammonia alkalizing agents such as from the ColourTouch® range marketed by Wella, INOA® range marketed by L'Oréal or Essensity® range marketed by Schwarzkopf. The components may for example be formulated and delivered as aqueous hair product, emulsion, gel, aerosol, or foam.

The hair colouring compositions of the presently claimed invention may further comprise any usual hair colouring chassis ingredients and use any common ingredients, known in the field as long as they are compatible with the requirements set in the claims.

### Alkalizing Agent

The hair colouring composition of the presently claimed invention comprises
a) at least one alkalizing agent of the general formula (I),

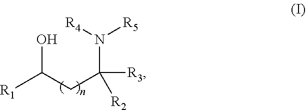

wherein
R1 is hydrogen or linear or branched C1-C6 alkyl which is unsubstituted or substituted with 1, 2, 3 or 4 OH,
R2 is hydrogen or linear or branched C1-C6 alkyl which is unsubstituted or substituted with 1 or 2 OH,
R3 is hydrogen or linear or branched, unsubstituted C2-C6 alkyl,
or
R2 and R3 together with the carbon atoms to which they are bonded form a saturated 5-, 6-, 7-or 8-membered carbocyclic ring that is unsubstituted or substituted with 1, 2, 3, or 4 substituents selected from the group consisting of OH and NH₂,
R4 is hydrogen or linear or branched, unsubstituted C1-C6 alkyl,
R5 is hydrogen or linear or branched, unsubstituted C1-C6 alkyl, and
n is 0, 1, 2 or 3.

In one embodiment of the presently claimed invention,
R1 is hydrogen or linear or branched C1-C4 alkyl which is unsubstituted or substituted with 1, 2, 3 or 4 OH,
R2 is hydrogen or linear or branched C1-C4 alkyl which is unsubstituted or substituted with 1 OH,
R3 is hydrogen,
or
R2 and R3 together with the carbon atoms to which they are bonded form a saturated 6-membered carbocyclic ring that is unsubstituted or substituted with 1, 2, 3, or 4 substituents selected from the group consisting of OH and NH₂,
R4 is hydrogen or linear or branched, unsubstituted C1-C3 alkyl,
R5 is hydrogen or linear or branched, unsubstituted C1-C3 alkyl, and
n is 0, 1 or 2.

The alkalizing agent of formula (I) is not monoethanolamine.

For the purposes of the presently claimed invention, the term “alkyl” covers acyclic saturated hydrocarbon residues, which may be branched or linear and unsubstituted or substituted with, as in the case of C1-C6 alkyl, 1 to 6 (i.e. 1, 2, 3, 4, 5 or 6) carbon atoms or with, as in the case of C1-C4 alkyl, 1 to 4 (i.e. 1, 2, 3 or 4) carbon atoms. In a preferred embodiment, the unsubstituted linear C1-C6 alkyl is preferably selected from the group consisting of methyl, ethyl, propyl, butyl, pentyl and hexyl. In a preferred embodiment, the unsubstituted branched C1-C6 alkyl is preferably selected from the group consisting of isopropyl, iso-butyl and neo-pentyl.

For the purpose of the presently claimed invention, “carbocyclic ring” refers to a ring, wherein the atoms forming the ring backbone are selected from carbon atoms only.

In one embodiment, the at least one alkalizing agent of general formula (I) is preferably selected from the group consisting of 2-amino-1-propanol, 1-amino-2-propanol, 3-amino-1-propanol, 2-amino-propane-1,3-diol, 1-amino-2-butanol, 4-amino-2-butanol, 4-amino-1-butanol, 3-amino-1-butanol, 3-amino-2-butanol, 2-amino-1-butanol, dimethyl glucamine, 2,5-diaminocyclohexane-1,4-diol, 2-aminocycohexan-1-ol and 2,4,6-triaminocyclohexane-1,3,5-triol; more preferably from the group consisting of 2-amino-1-propanol, 1-amino-2-propanol, 2-amino-1-butanol and dimethyl glucamine; even more preferably from the group consisting of 2-amino-1-propanol and 2-amino-1-butanol.

2-Amino-1-propanol is depicted below. 2-Amino-1-propanol is also sometimes referred to as Alaninol or is abbreviated as 2A1P. Alanilol is a biodegradable material which is supplied from Angus.

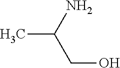

2-Amino-1-propanol exists in the form of its(S)-and (R)-enantiomer and can be obtained in the form of a pure enantiomer or in the form of a racemic mixture. In one embodiment of the presently claimed invention, either one of the enantiomers or a mixture of enantiomers can be used. The racemic mixture is preferably used because it is more readily available and less expensive to synthesise.

In one embodiment, the component a) is preferably present in an amount in the range of ≥0.05 wt. % to ≤5.0 wt. %, more preferably in an amount in the range of ≥0.05 wt. % to ≤4.0 wt. %, even more preferably in an amount in the range of ≥0.1 wt. % to ≤3.0 wt. %, most preferably in an amount in the range of ≥0.1 wt. % to ≤2.5 wt. %, based on the total weight of the oxidative hair colouring composition.

In another embodiment of the presently claimed invention, the ready to use composition may comprise a second alkalizing agent selected from the group consisting of monoethanolamine and ammonia in combination with at least one alkalizing agent of general formula (I). Monoethanolamine and/or ammonia may be present in the same composition as the at least one alkalizing agent of general formula (I), or they may be present in a separate composition which is mixed along with the oxidizing composition to create the colouring composition.

In one embodiment, the oxidative hair colouring composition comprises ammonia to component a) in a molar ratio in the range of 5.0:1.0 to 1.0:5.0, preferably in the range of 3.0:1.0 to 1.0:3.0, more preferably in the range of 2.0:1.0 to 1.0:2.0, most preferably in the range of 1.5:1.0 to 1.0:1.5.

In one embodiment, the oxidative hair colouring composition comprises ammonia to 2-amino-1-propanol in a molar ratio in the range of 5.0:1.0 to 1.0:5.0, preferably in the range of 3.0:1.0 to 1.0:3.0, more preferably in the range of 2.0:1.0 to 1.0:2.0, most preferably in the range of 1.5:1.0 to 1.0:1.5.

In one embodiment, the oxidative hair colouring composition comprises ammonia to 2-amino-1-butanol in a molar ratio in the range of 5.0:1.0 to 1.0:5.0, preferably in the range of 3.0:1.0 to 1.0:3.0, more preferably in the range of 2.0:1.0 to 1.0:2.0, most preferably in the range of 1.5:1.0 to 1.0:1.5.

In one embodiment, the oxidative hair colouring composition comprises monoethanolamine to component a) in a molar ratio in the range of 5.0:1.0 to 1.0:5.0, preferably in the range of 3.0:1.0 to 1.0:3.0, more preferably in the range of 2.0:1.0 to 1.0:2.0, most preferably in the range of 1.5:1.0 to 1.0:1.5.

In one embodiment, the oxidative hair colouring composition comprises monoethanolamine to 2-amino-1-propanol in a molar ratio in the range of 5.0:1.0 to 1.0:5.0, preferably in the range of 3.0:1.0 to 1.0:3.0, more preferably in the range of 2.0:1.0 to 1.0:2.0, most preferably in the range of 1.5:1.0 to 1.0:1.5.

In one embodiment, the oxidative hair colouring composition comprises monoethanolamine to 2-amino-1-butanol in a molar ratio in the range of 5.0:1.0 to 1.0:5.0, preferably in the range of 3.0:1.0 to 1.0:3.0, more preferably in the range of 2.0:1.0 to 1.0:2.0, most preferably in the range of 1.5:1.0 to 1.0:1.5.

### Oxidizing Agent

The oxidizing agents may be water-soluble inorganic peroxide materials which are capable of yielding hydrogen peroxide in an aqueous solution.

In one embodiment of the presently claimed invention, the oxidizing agents are selected from the group consisting of hydrogen peroxide, inorganic alkali metal peroxides (such as sodium periodate and sodium peroxide), organic peroxides (such as urea peroxide, melamine peroxide), inorganic perhydrate salt bleaching compounds (such as the alkali metal salts of perborates, percarbonates, perphosphates, persilicates, persulphates), and mixtures thereof; preferably from the group consisting of hydrogen peroxide, persulphates, and mixtures thereof; most preferably the oxidizing agent is hydrogen peroxide.

“Water-soluble,” as defined herein, means that in standard condition at least 0.1 g, 1 g, or 10 g of the oxidizing agent can be dissolved in 1 litre of deionized water.

In one embodiment, the component b) is preferably present in an amount in the range of ≥0.7 wt. % to ≤8.0 wt. %, more preferably in an amount in the range from of ≥0.9 wt. % to ≤6.0 wt. %, more preferably in an amount in the range from of ≥0.9 wt. % to ≤4.0 wt. %, based on the total weight of the oxidative hair colouring composition.

The oxidizing agents can be provided in aqueous solution or as a powder which is dissolved prior to use.

### Buffering Agents and Buffering System

The hair colouring compositions comprises at least one buffering agent which forms the buffering system in the composition. The buffering system helps to maintain the pH of the compositions less than 9. In a more preferred embodiment, the buffering system helps to maintain the pH of the hair colouring compositions in the range from ≥6.5 to ≤8.5.

In one embodiment of the presently claimed invention, the buffering agent is comprised in at least one of the tint composition (A), the oxidizing composition (B) or at least in one gel network thickener system.

In another embodiment of the presently claimed invention, the buffering agent is comprised in both the tint composition (A), the oxidizing composition (B) and in the gel network thickener system.

In one aspect of the presently claimed invention, the buffering system comprises at least one buffering acidic compound, and/or at least one buffering alkali compound.

In one embodiment of the presently claimed invention, the buffering acidic compound is selected from the group consisting of organic and inorganic acids; e.g., from the group consisting in sulfurous acid, sulphuric acid, hydrochloric acid, hyponitrous acid, nitrous acid, nitric acid, phosphoric acid, phosphorous acid, citric acid, malic acid, and their mixtures; or from the group consisting in phosphoric acid, phosphorous acid, citric acid, malic acid, hydrochloric acid, hyponitrous acid and mixtures thereof.

In a preferred embodiment the buffering acidic compound is selected from phosphoric acid, hydrochloric acid, hyponitrous acid, and mixtures thereof, more preferably the buffering acidic compound is phosphoric acid.

In one embodiment of the presently claimed invention, the buffering alkali compound is selected from the group consisting of alkali metal salts, amino acids, and salts thereof; or from the group consisting of glycine, alkali metal salts, amino acids, chlorides, nitrates and salts thereof. In a preferred embodiment, the buffering alkali compound is selected from alkali metals of chlorides, nitrates and/or phosphoric acid, glycine and salts thereof. In a more preferred embodiment, the buffering alkali compound is selected from alkali metal of phosphoric acid and salts thereof; or disodium phosphate and potassium chloride.

In another aspect of the presently claimed invention, the buffering system comprises a buffering acidic compound in combination with the corresponding buffering alkali compound.

The combination of the buffering acidic compound and its buffering alkali compound may be selected from the group consisting of phosphoric acid/potassium dihydrogen phosphate, phosphoric acid/sodium dihydrogen phosphate, phosphoric acid/dipotassium hydrogen phosphate, phosphoric acid/disodium hydrogen phosphate, phosphoric acid/potassium phosphate, phosphoric acid/sodium phosphate, hydrochloric acid/potassium chloride, hydrochloric acid/potassium hydrogen phthalate, hydrochloric acid/sodium citrate, hydrochloric c acid/potassium dihydrogen citrate, citric acid/dihydrogen citrate, citric acid/sodium citrate or citric acid/disodium hydrogen phosphate.

In a preferred embodiment the buffering acidic compound is phosphoric acid and the corresponding buffering alkali compound is disodium phosphate. Alternatively, the buffering system comprises a buffering acidic compound being phosphoric acid, and a corresponding buffering alkali compound being glycine; or a buffering acidic compound being citric acid, and a corresponding buffering alkali compound being disodium phosphate; or a buffering acidic compound being citric acid, and a corresponding buffering alkali compound being glycine.

In one embodiment of the present invention, the buffering system comprises from ≥0.05 wt. % to ≤4.0 wt. % phosphoric acid and from ≥0.05 wt. % to ≤3.5 wt. % disodium phosphate, based on the total weight of the oxidizing composition (B).

In a preferred embodiment of the presently claimed invention, the ratio of phosphoric acid to disodium phosphate is in the range from 1.0:1.0 to 10.0:1.0, more preferably in the range from 1.0:1.0 to 5.0:1.0, even more preferably in the range from 1.0:1.0 to 2.0:1.0.

In another embodiment of the presently claimed invention, the buffering system comprises from ≥0.05 wt. % to ≤4.0 wt. % of phosphoric acid more preferably from ≥0.08 wt. % to ≤3.0 wt. % of phosphoric acid, based on the total weight of the oxidizing composition (B) and from ≥0.05% to ≤3.5%, more preferably from ≥0.08% to 2.0% disodium phosphate, based on the total weight of the oxidizing agent; wherein the ratio of phosphoric acid to disodium phosphate is in the range from 1.0:1.0 to 10.0:1.0, more preferably in the range from 1.0:1.0 to 5.0:1.0, even more preferably in the range from 1.0:1.0 to 2.0:1.0.

In one embodiment, the oxidative hair colouring composition has a pH in the range from ≥6.0 to ≤9.0, more preferably in the range from ≥6.5 to ≤8.5.

Oxidative Primary Dye Precursors

In one embodiment of the presently claimed invention, the oxidative primary dye precursors are selected from of the group consisting of toluene-2,5-diamine, p-phenylenediamine, n-phenyl-p-phenylenediamine, N,N-bis(2-hydroxyethyl)-p-phenylenediamine, hydroxyethyl-p-phenylenediamine sulphate, hydroxypropyl bis(n-hydroxyethyl-p-phenylenediamine), 2-methoxymethyl-p-phenylenediamine, 2-chloro-p-phenylenediamine, p-aminophenol, p-methylaminophenol, 4-amino-m-cresol, 6-amino-m-cresol, bis(5-amino-2-hydroxyphenyl)methane, tetraaminopyrimidine, 2,5,6-triamino-4-pyrimidinol, 4,5-diamino-1-(2-hydroxyethyl)pyrazol 2,3-diaminodihydroxypyrazolopyrazolone dimethosulfonate, 4,5-diamino-1-hexylpyrazol, hydroxypropyl-p-phenylenediamine, dimethylpiperazinium aminopyrazolopyridine chloride hydrochloride, methylimidazoliumpropyl p-phenylenediamine, hydroxyethoxy aminopyrazolopyridine and salts thereof. Preferably, the oxidative primary dye precursors are selected from of the group consisting of toluene-2,5-diamine, 4,5-diamino-1-hexylpyrazol, N,N-bis(2-hydroxyethyl)-p-phenylenediamine, hydroxypropyl-p-phenylenediamine, 2-methoxymethyl-p-phenylenediamine and salts thereof.

### Oxidative Coupler Dye Precursors

In one embodiment of the presently claimed invention, the oxidative coupler dye precursors are selected from the group consisting of resorcinol, 4-chlororesorcinol, 2-chlororesorcinol, 2-methylresorcinol, m-aminophenol, 4-amino-2-hydroxytoluene, 2-methyl-5-hydroxyethylaminophenol, 3-amino-2,6-dimethylphenol, 3-amino-2,4-dichlorophenol, 5-amino-6-chloro-o-cresol, 5-amino-4-chloro-o-cresol, hydroxybenzomorpholine, 2-amino-5-ethylphenol, 6-amino-m-cresol, 6-amino-o-cresol, 2,4-diaminophenoxyethanol, 2-amino-4-hydroxyethylaminoanisole, 1,3-bis-(2,4-diaminophenoxy)propane, 2,6-dihydroxyethylaminotolueneu, m-phenylenediamine, 2,4-diamino-1,5-di(2-hydroxyethoxybenzene, 1-naphthol, 2-methyl-1-naphthol, 1,5-naphthalenediol, 2,7-naphthalenediol, 1-acetoxy-2-methylnaphthalene, 2,6-dihydroxy-3,4-dimethylpyridine, 2,6-dimethoxy-3,5-pyridinediamine, 3-amino-6-methoxy-2-(methylamino)-pyridine, 2-amino-3-hydroxypyridine, 2,6-diaminopyridine, dihydroxyindole, 6-hydroxyindole, dihydroxyindoline, phenyl methyl pyrazolone, 1,2,4-trihydroxybenzene, 5-((2-hydroxyethyl)amino)-1,3-benzodioxol, isatin, hydroquinone, 4-formyl-1-methylquinolinium-p-toluenesulfonate and salts thereof. Preferably, the oxidative coupler dye precursors are selected from the group consisting of resorcinol, 2-methylresorcinol, 4-chlororesorcinol, 2-chlororesorcinol, m-aminophenol, hydroxybenzomorpholine, 2,4-diaminophenoxyethanol, 4-amino-2-hydroxytoluene, 2-methyl-5-hydroxyethylaminophenol, 5-((2-hydroxyethyl)amino)-1,3-benzodioxol and salts thereof.

These and other primary dye precursors and coupler dye precursors may be used in different combination to achieve the nuance sought, as is known in the art.

### Direct Dyes

Direct dyes may also be incorporated in any of components of the presently claimed invention, in particular the tint composition. The compositions of the presently claimed invention may also comprise compatible direct dyes, in an amount sufficient to provide additional colouring, particularly with regard to intensity. Typically, such an amount will range from 0.005% to 4%, by weight, of the composition. When the composition is obtained by mixing a tint composition and an oxidizing composition, the direct dyes are usually incorporated in the tint composition.

In one embodiment of the presently claimed invention, the direct dye is selected from the list consisting of Acid Yellow 1, Disperse Red 17, Basic Brown 17, Acid Yellow 9, Pigment Red 49, Acid Yellow 11, Acid Black 1, 4-Nitro-1,2-phenylenediamine, Picramic acid, HC Red 13, N,N′-Bis(2-hydroxyethyl)-2-nitro-p-phenylenediamine, HC Yellow 5, HC Red 7, HC Blue 2, HC Yellow 4, HC Yellow 3, HC Blue 1, HC Yellow 2, HC Orange 1, HC Red 1, HC Red 3, 4-Amino-3-nitrophenol, 2-Hydroxyethylamino-5-nitroanisole, 3-Nitro-p-hydroxyethylaminophenol, 3-Methylamino-4-nitrophenoxyethanol, 2-Nitro-5-glyceryl methylaniline, HC Violet 1, HC Orange 2, HC Orange 3, HC Yellow 9, 4-Nitrophenyl aminoethylurea, HC Red 10, HC Red 11, 2-Hydroxyethyl picramic acid, HC Blue 12, HC Yellow 6, Hydroxyethyl-2-nitro-p-toluidine, HC Yellow 12, HC Blue 11, HC Blue 10, HC Blue 6, HC Yellow 7, HC Yellow 10, HC Blue 9, HC Blue 13, 2-Chloro-6-(ethylamino)-4-nitrophenol, 6-Nitro-2,5-pyridinediamine, HC Violet 2, 2-Amino-6-Chloro-4-Nitrophenol, 4-Hydroxypropylamino-3-nitrophenol, Acid Blue 7, HC Yellow 13, HC Red 14, HC Yellow 15, HC Yellow 14, 2,6-Diamino-3-((pyridin-3-yl) azo) pyridine, Basic Orange 69, HC Red 16, Basic violet 2, Basic Red 51, Basic Yellow 87, Basic Orange 31, HC Blue 16, HC red 17, HC Yellow 17, HC blue 18, HC Yellow 16, HC Red 18, HC Orange 6, Basic Orange 1, Basic Red 76, Basic Brown 16, Basic Yellow 57, CI14700, Acid Red 14, Acid Orange 7, Acid Red 88, FD&C Yellow 6, Acid Red 27, Acid Orange 10, Acid Red 33, Acid red 155, Acid Yellow 23, Food Black 2, CI 28440, HC Brown 2, Acid Blue 1, Acid Blue 3, Acid Blue 9, Acid Violet 49, CI 42735, Basic Blue 26, Acid violet 9, Acid Red 92, Acid Yellow 3, Beta-Carotene, CI 50420, Basic Blue 99, 1-Amino-4-hydroxy-9, 10-anthracendion, CI 60725, Acid violet 43, Disperse Violet 1, Acid blue 62, Copper phthalocyanine, Carbon black, Disperse Black 9, Lycopene, Hydroxyanthraquinoneaminopropyl methyl morpholinium methosulfate, HC Blue 8, HC Green 1, Curry red, 2-Hydroxy-1,4-naphthoquinone, CI 77289, Solvent Green 7, Lawsonia Inermis Cera, CI 73000, Indigofera tinctoria, Carmines, HC Blue 14, Acid Red 18, CI 42053, Acid Red 52, Acid Green 25, Disperse Blue 377, Pigment Red 57, HC Blue 15, Tetrabromphenol Blue, Cationic Blue 347, Bromthymol blue sodium, Anthocyanins, Chlorophyllin-Copper complex, Annatto, Natural Green 3, Betanin, Capsanthin, Basic Yellow 29 and combinations thereof.

### Chelants

Chelants may be used prior to application of a hair colouring composition to remove and/or bind metal ions which are bonded to hair. For example, US 5,635,167 discloses a process for the removal of exogenous metal ions that are attached to hair. The treatment comprises contacting hair with a blend of chelating agents (selected from the group consisting of amino acid chelating agents, polyphosphate chelating agents and phosphonate chelating agents) at a pH of between 4 and 9 and at a concentration of between 4% to 25% by weight prior to colouring.

As already disclosed in WO 02/089754, among the usual ingredients encountered within a hair colouring composition, the presence of a chelant such as EDTA or EDDS and their salts thereof may be beneficial in several ways, as chelants can reduce hair damage caused by oxidizing agents.

Chelants, especially disodium EDTA, are also commonly used as stabilizer in the oxidizing component. Besides EDDS and EDTA, any other chelants can be used in the presently claimed invention, such as; diethylenetriamine penta (methylene phosphonic acid) DTPMP, methylglycinediacetic acid trisodium salt (MGDA), 1-hydroxyethylidene-1,1-diphosphonic acid (HEDP), EDDG, HPDDS and EDDHA and their salts thereof.

In a further embodiment of the presently claimed invention, the at least one chelant is selected from the group consisting of ethylenediamine tetraacetic acid (EDTA), ethylenediamine-N,N′-disuccinic acid (EDDS), ethylenediamine-N,N′-diglutaric acid (EDDG), 2-hydroxypropylenediamine-N,N′-disuccinic acid (HPDDS), glycinamide-N,N′-disuccinic acid (GADS), ethylenediamine-N-N′-bis(ortho-hydroxyphenyl acetic acid) (EDDHA), dimethyl glucamine (DMG), salts thereof, derivatives thereof, and mixtures thereof.

In one embodiment of the presently claimed invention, the chelant is EDDS, more preferably is (S,S)-EDDS and their salts thereof.

In a further embodiment of the presently claimed invention, the hair colouring composition comprises at least one chelant in an amount in the range from ≥0.20 wt. % to ≤1.5 wt. %, based on the total weight of the oxidative hair colouring composition.

### Fatty Substances

In one embodiment of the presently claimed invention, the hair colouring composition may comprise at least one fatty substance that does not contain any free carboxylic acid groups, i.e. free of —C(═O)—OH groups and —C(═O)—O. The term “fatty substance” means an organic compound that is insoluble in water at room temperature (25° C.) and at atmospheric pressure (≤15 mg/L water, preferably ≤5 mg/L water). In addition, under the same temperature and pressure conditions, the fatty substances are soluble in organic solvents such as chloroform, ethanol or benzene.

In a preferred embodiment of the presently claimed invention, the fatty substances are selected from the group consisting of liquid hydrocarbons, non-silicone oils of animal, plant, mineral or synthetic origin, fatty alcohols, fatty acids, fatty acid esters and/or fatty alcohol esters, non-silicone waxes, and silicones or mixtures thereof.

More particularly, the liquid hydrocarbons are selected from the group consisting of:
linear or branched, optionally cyclic, C6-C16 alkanes such as hexane, undecane, dodecane, tridecane, and isoparaffins, such as isohexadecane, isododecane and isodecane,
linear or branched hydrocarbons of mineral, animal or synthetic origin with more than 16 carbon atoms, such as volatile or non-volatile liquid paraffins and derivatives thereof, petroleum jelly, liquid petroleum jelly, polydecenes, hydrogenated polyisobutene such as Parleam®, and squalane.

In a preferred embodiment of the presently claimed invention, the liquid hydrocarbons are selected from the group consisting of volatile or non-volatile liquid paraffins, and liquid petroleum jelly.

The compositions of the presently claimed invention preferably comprise an amount of fatty substances that do not contain any free carboxylic acid groups of ≥2 wt. %, more preferably ≥10 wt. %, even more preferably ≥20 wt. %, most preferably ≥30 wt. %, based on the total weight of the hair colouring composition.

In a further embodiment of the presently claimed invention, the hair colouring composition comprises fatty substances in an amount in the range from ≥2.0 wt. % to ≤70.0 wt. %, based on the total weight of the oxidative hair colouring composition.

### Fatty Alcohol

In one embodiment of the presently claimed invention, a creamy carrier for the tint composition (A) preferably comprises from ≥10.0 wt. % to ≤30 wt. % of at least one fatty alcohol with 10 to 24 carbon atoms; and/or one or more of ≥0.2 wt. % to ≤6.0 wt. % of at least one diester of formula: R¹—CO—O—(CH2-CH2-O)ₙ—CO—R², where n is 1, 2 or 3, and R1 and R2 are the same or different alkyl radicals with 12 to 20 carbon atoms; and/or one or more of ≥0.5 wt. % glycerine fatty acid ester with 10 to 24 carbon atoms; and/or one or more of ≥0.1 wt. % to ≤10 wt. % of non-ionic and/or anionic and/or ampholytic emulsifiers, based on the total weight of the tint composition.

Lower levels of fatty alcohol can also be used in this chassis, if a less thick composition is desired, for example a level of from ≥2.0 wt. % to ≤10 wt. % of at least one fatty alcohol with 10 to 24 carbon atoms can be used.

The formulations disclosed in WO 98/11863A2 may also be used as a reference for the presently claimed invention. The formulations disclosed in this reference document contain a beeswax-protein hydrolysate-and/or amino acid association, which however may or may not be present in the tint component of the presently claimed invention.

In one embodiment of the presently claimed invention, the hair colouring composition may be a three-component system, as exemplified in US 2010/0223739 A2, in which case the aqueous cosmetic composition comprising at least one fatty substance and at least one surfactant as defined in this document may be considered as a third composition.

### Surfactants

In one embodiment of the presently claimed invention, the hair colouring composition comprises at least one surfactant. The at least one surfactant is preferably anionic, cationic or non-ionic.

The at least one surfactant is preferably at least one non-ionic surfactant which is selected from the group consisting of fatty alcohol polyoxyalkylene esters, alkyl polyoxyalkylene ethers which are derived from C1-C6-alcohols or from C7-C30-fatty alcohols, alkylaryl alcohol polyoxyethylene ethers, alkoxylated animal and/or plant fats and/or oils, glycerol esters, alkylphenol alkoxylates, fatty amine alkoxylates, fatty acid amide and fatty acid diethanolamide alkoxylates, ethoxylates thereof, sugar surfactants, sorbitol esters, polyoxyethylene sorbitan fatty acid esters, alkyl polyglycosides, N-alkylgluconamides, alkyl methyl sulfoxides and alkyl dimethyl phosphine oxides. In one embodiment, the alkyl polyoxyalkylene ethers are selected from the group consisting of steareth-20, steareth-100 and steareth-150.

### Thickeners

The colouring compositions of the presently claimed invention preferably comprises a thickener, in particular a polymeric thickener in an amount that is sufficient to impart a viscosity to the composition that allows for its ready application to hair without unduly dripping off the hair, as is known in the art. Typically, such an amount will be at least about 0.1 wt. %, in some embodiments, at least about 0.5 wt. %, in other embodiments, at least about 1.0 wt. %, based on the total weight of the colouring composition.

Examples of commonly used associative polymeric thickeners are sold under the tradename Aculyn-22 and Aculyn-33 by the company Rohm & Haas, Permulen TR1, Carbopol 2020, Carbopol Ultrez-21 by the company Noveon, and Structure 2001 and Structure 3001 by the company National Starch. Other suitable polymers include polyether polyurethanes, for example Aculyn-44 and Aculyn-46 by the company Rohm and Haas. Another suitable associative polymer is cellulose modified with groups comprising at least one C8-C30 fatty chain, such as the product Natrosol Plus Grade 330 CS sold by the company Aqualon.

In one embodiment of the presently claimed invention the hair colouring composition comprises salt tolerant thickeners, including but not limited to: xanthan, guar, hydroxypropyl guar, scleroglucan, methyl cellulose, ethyl cellulose (available as AQUACOTE®, hydroxyethyl cellulose (NATROSOL®), carboxymethyl cellulose, hydroxypropylmethyl cellulose, microcrystalline cellulose, hydroxybutylmethyl cellulose, hydroxypropyl cellulose (available as KLUCEL®), hydroxyethyl ethyl cellulose, cetyl hydroxyethyl cellulose (available as NATROSOL® Plus 330), N-vinylpyrrolidone (available as POVIDONE®), Acrylates/Ceteth-20 Itaconate Copolymer (available as STRUCTURE® 3001), hydroxypropyl starch phosphate (available as STRUCTURE® ZEA), polyethoxylated urethanes or polycarbamyl polyglycol ester (e.g. PEG-150/Decyl/SMDI copolymer (e.g. ACULYN® 44), PEG-150/Stearyl/SMDI copolymer (available as ACULYN® 46), trihydroxystearin (available as THIXCIN®), acrylates copolymer (e.g. available as ACULYN® 33) or hydrophobically modified acrylate copolymers (e.g. Acrylates/Steareth-20 Methacrylate Copolymer (available as ACULYN® 22), acrylates/steareth-20 methacrylate crosspolymer (available as ACULYN® 88), acrylates/vinyl neodecanoate crosspolymer (available as ACULYN® 38), acrylates/beheneth-25 methacrylate copolymer (available as ACULYN® 28), acrylates/C 10-30 alkyl acrylate crosspolymer (available as Carbopol® ETD 2020), non-ionic amphophilic polymers comprising at least one fatty chain and at least one hydrophilic unit selected from polyether urethanes comprising at least one fatty chain.

In one embodiment of the presently claimed invention, the hair colouring composition comprises at least one mineral thickener selected from organophilic clays, fumed silicas, or mixtures thereof. The at least one mineral thickener is present in an amount ranging from ≥0.05 wt. % to ≤30 wt. %, based on the total weight of the colouring composition.

In one embodiment of the presently claimed invention, the organic thickener is selected from cellulose-based thickeners (hydroxyethycellulose, hydroxypropyl cellulose, carboxymethylcellulose), guar gum and derivatives thereof (hydroxypropyl guar), gums of microbial origin (xanthan gum, scleroglucan gum), crosslinked homopolymers of acrylic acid or of acrylamido-propanesulfonic acid, and cellulose-based thickeners, such as hydroxyethylcellulose. The at least one organic thickener is present in an amount ranging from ≥0.1 wt. % to ≤20 wt. %, based on the total weight of the colouring composition.

### Gel Network Thickener System

In one embodiment of the presently claimed invention, the hair colouring composition preferably comprises a gel network thickener system. The gel network thickener system is typically provided in the tint composition and subsequently mixed with the oxidizing composition whilst retaining the gel like network system in the resultant mixed colouring composition. Such gel network thickener systems comprise a surfactant and a fatty alcohol.

In one embodiment of the presently claimed invention, the gel network system preferably comprises a linear or branched C14 to C30 fatty alcohol, preferably selected from the group consisting of cetyl alcohol, stearyl alcohol, cetostearyl alcohol or behenyl alcohols or mixtures thereof. According to the presently claimed invention the tint composition preferably comprises from ≥1.0 wt. % to 18.0 wt. %, more preferably from ≥2.0 wt. % to ≤12.0 wt. % of fatty alcohol.

In one embodiment of the presently claimed invention, the gel network system preferably comprises an ionic, cationic or non-ionic surfactant or amphophile.

The gel network thickener system preferably comprises at least one anionic surfactant selected from C8 to C30 alkyl phosphate, C8 to C30 alkyl ether phosphate or mixtures thereof, and a C14 to C30 fatty alcohol. Other anionic gel network forming surfactants may also be used.

The gel network thickener system preferably comprises at least one non-ionic surfactant selected from the group consisting of fatty alcohol polyoxyalkylene esters, alkyl polyoxyalkylene ethers which are derived from C7-C30-fatty alcohols, alkylaryl alcohol polyoxyethylene ethers, alkoxylated animal and/or plant fats and/or oils, glycerol esters, alkylphenol alkoxylates, fatty amine alkoxylates, fatty acid amide and fatty acid diethanolamide alkoxylates, ethoxylates thereof, sugar surfactants, sorbitol esters, polyoxyethylene sorbitan fatty acid esters, alkyl polyglycosides, N-alkylgluconamides, alkyl methyl sulfoxides and alkyl dimethyl phosphine oxides.

Representative examples of anionic surfactants include salts (such as alkaline salts, for example, sodium salts, ammonium salts, amine salts, amino alcohol salts and magnesium salts) of the following compounds: alkyl ether carboxylates, alkyl ether sulphates, alkyl glyceryl sulphonates, alkylamido ether sulphates, alkylarylpolyether sulphates, alkyl monoglyceride sulphates, alkyl ether sulphonates, alkylamide sulphonates; alkyl succinates, alkyl sulphosuccinates, alkyl ether sulphosuccinates, alkylamide sulphosuccinates; alkyl sulphosuccinamates; alkyl sulphoacetates; alkyl ether phosphates; acyl sarcosinates, N-acyl methylaminopropionate; acyl isethionates, N-acyltaurates; acyl lactylates; carboxyalkyl ether of alkyl polyglucosides; alkyl lecithin derivatives. The alkyl or acyl radical of all of these various compounds, for example, comprises from about 8 to 30 carbon atoms, and the aryl radical, for example, is chosen from phenyl and benzyl groups.

Anionic surfactants include alkyl ether phosphates, alkyl ether sulphates, alkyl glyceryl sulphonates, N-acyl sarcosinates, N-acyl taurates, acyl lactylates and carboxyalkyl ether of alkyl polyglucosides. Yet more preferable surfactants are selected from alkyl ether phosphates having an average 1 to 20, 1 to 10 or 1 to 3 ethylene oxide units.

The gel network thickener system preferably comprises at least one cationic surfactant selected from quaternary ammonium salts or amido-amines having at least one fatty chain containing at least about 20 carbon atoms and mixture thereof.

The quaternary ammonium salts have general formula N⁺(R¹R²R³R⁴)X⁻: wherein, R¹ is selected from linear and branched radicals comprising about 20 to 30 carbon atoms, R² is selected from linear and branched radicals comprising about 20 to 30 carbon atoms or the same group as radicals R³ to R⁴, the radicals R³ to R⁴, which can be identical or different, are selected from linear and branched aliphatic radicals comprising from about 1 to 4 carbon atoms, and aromatic radicals such as aryl and alkylaryl, the aliphatic radicals can comprise at least one hetero atom such as oxygen, nitrogen, sulphur and halogens, the aliphatic radicals are chosen, for example, from alkyl, alkoxy and alkylamide radicals, and wherein X⁻ is an anion selected from halides, such as chloride, bromide and iodide) (C₂-C₆) alkyl sulphates, such as methyl sulphate, phosphates, alkyl and alkylaryl sulphonates, and anions derived from organic acids, such as acetate and lactate. The cationic surfactant is, for example, a behentrimonium chloride, behenamidopropyltrimonium methosulfate, stearamidopropyltrimonium chloride, arachidtrimonium chloride and mixtures thereof.

The amido-amine have general formula R′¹—CONH(CH₂)ₙNR′²R′³: wherein, R′¹ is selected from linear and branched radicals comprising about 20 to 30 carbon atoms, the radicals R′² and R′³, which can be identical or different, are selected from hydrogen, linear and branched aliphatic radicals comprising from about 1 to 4 carbon atoms, and aromatic radicals such as aryl and alkylaryl, the aliphatic radicals can comprise at least one hetero atom such as oxygen, nitrogen, sulphur and halogens, the aliphatic radicals are chosen, for example, from alkyl, alkoxy and alkylamide radicals, and wherein n is integer from 1 to 4. The amido-amine can be selected from, for example, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, and mixtures thereof.

In one preferred embodiment of the presently claimed invention, the surfactant which is used to form the gel network systems is selected from the group consisting of ceteareth-25, steareth-20, steareth-100, steareth-150, steareth-200 and mixtures thereof. These surfactants act as a co-emulsifier and stabilizer of the gel network system.

Those skilled in the art will recognize that gel network thickener systems usually have a complex structure of networked lamellar bi-layers and/or vesicles and sometimes crystals. These systems usually have creamy appearance and feel and are thus particularly desirable.

In one embodiment of the presently claimed invention, the hair colouring formulation preferably further comprises at least one anionic, cationic, nonionic, amphoteric, or zwitterionic polymer or mixtures thereof; antioxidants; penetrants; sequestrants; fragrances; dispersants; film-forming agents; ceramides; preserving agents; and opacifiers.

In a preferred embodiment of the presently claimed invention the suitable non-associative cross-linked polycarboxylic polymers for use herein can be selected, for example, from: (i) cross-linked acrylic acid homopolymers; or (ii) copolymers of acrylic or (meth) acrylic acid and of C1-C6 alkyl acrylate or (meth) acrylate. Such polymers are sold under the names Carbopol 980, 981, 954, 2984, 5984 by the company Noveon or Synthalen M, Synthalen L and Synthalen K by the company 3V Sigma, or Aculyn-33 by the company Rohm and Haas.

Polysaccharides may also be used, for example, glucans, modified and unmodified starches, amylose, amylopectin, glycogen, dextrans, celluloses and derivatives thereof (methylcelluloses, hydroxyalkylcelluloses, ethyl hydroxyethylcelluloses, and carboxymethylcelluloses), mannans, xylans, lignins, arabans, galactans, galacturonans, chitin, chitosans, glucuronoxylans, arabinoxylans, xyloglucans, glucomannans, pectic acids and pectins, alginic acid and alginates, arabinogalactans, carrageenans, agars, glycosaminoglucans, gum arabics, gum tragacanths, ghatti gums, karaya gums, carob gums, galactomannans, such as guar gums, and nonionic derivatives thereof (hydroxypropyl guar) and bio-polysaccharides, such as xanthan gums, gellan gums, welan gums, scleroglucans, succinoglycans and mixtures thereof. Suitable polysaccharides are described in “Encyclopedia of Chemical Technology”, Kirk-Othmer, Third Edition, 1982, volume 3, pp. 896-900, and volume 15, pp. 439-458, in “Polymers in Nature” by E. A. MacGregor and C. T. Greenwood, published by John Wiley & Sons, Chapter 6, pp. 240-328, 1980, and in “Industrial Gums—Polysaccharides and their Derivatives”, edited by Roy L. Whistler, Second Edition, published by Academic Press Inc.

The oxidizing composition of the presently claimed invention may be based on the same or similar (i.e. having the same ingredients but possibly at different level) formulation chassis as the tint component.

In one embodiment of the presently claimed invention, the colouring composition of the invention is obtained by mixing the tint composition and oxidizing composition. The tint composition comprises at least one alkalizing agent and a mixture of oxidative dyes. The oxidizing composition comprises the hydrogen peroxide. Such two component systems are commonly used in the art and each component is formulated so that the resulting mixture is a hair colouring composition according to the invention.

### Packaging

In one embodiment of the presently claimed invention, before use, the tint and oxidizing compositions used in the invention are normally packaged separately from one another. The compositions may be packaged in separate primary packages such as plastic bottle, sachet or tube. The components, in particular each component of a two-component composition, may however be packaged separately but within a common secondary package such as a carton or in different compartment of an aerosol or foam bottle, as in known in the trade. A conditioning composition, which can be applied after rinsing of the hair colouring composition, may also be packaged in such secondary package. On the other hand, the different components of the invention, in particular the third component, may be sold separately from the other components.

### Method of Hair Dying

Application of the hair colouring composition to the hair may be undertaken in several ways. Application of the hair colouring composition may take place on the whole head of hair of an end user. As used herein, the “whole head of hair” means that the hair all over the head from the root of the hair to the tip of the hair is included in the application process. By contrast, the application of the hair colouring composition may take place only on the root portion of the hair. The application to the root portion of the hair may still be over the entire head of the end user, but application of the hair colouring composition is applied only to the section of hair closest to the head (root portion), which is between about 0.01 mm to about 40 mm from the scalp of the head. After application to the root portion, product may be applied to the rest of the hair at a later stage to prevent over processing of the hair in the lengths and ends. Also, application may take place on a portion of hair. Application of a portion of hair is commonly referred to as highlighting or lowlighting. The portion of hair may be physically separated from the whole head of hair in a hair bundle or may be a smaller portion of hair than the whole head of hair. A hair bundle may be physically separated from a whole head of hair by a device including a plastic cap through which hair bundles are formed when hair is pulled through orifices in the plastic cap, metal foils encompassing a hair bundle, strand separators applied to hair at the root portion, or similar devices.

When present, an optional conditioning agent can be provided in an additional container. In the latter case, the conditioner can be mixed immediately before use and applied together with the other components, or the content of the additional container can be applied (after an optional rinse step) as a post-treatment immediately after the hair colouring composition.

According to one method for oxidatively colouring hair, the method comprises mixing a tint composition and an oxidizing composition and optionally a third component comprising a second non-ammonia alkalizing agent together to form a hair colouring composition, applying the hair colouring composition to the hair to form a treated hair surface, waiting for a period of 2-50 minutes, such as 3-25 minutes, and then removing the hair colouring composition from the treated hair surface through rinsing with water.

The methods of colouring hair also may further comprise working the hair colouring composition into the treated hair surface by hand or by a tool for a few minutes to ensure uniform application to the entire treated hair surface. The hair colouring composition remains on the treated hair surface while the end hair colour develops for a time period of 2 to 50 minutes to form oxidatively coloured hair. The consumer then rinses his/her oxidatively coloured hair thoroughly with tap water and allows it to dry and/or styles the oxidatively coloured hair.

In one embodiment, the presently claimed invention is directed to a hair colouring composition comprising,
a) ≥0.05 wt. % to ≤5.0 wt. %, more preferably ≥0.5 wt. % to ≤4.0 wt. %, of alkalizing agents selected from the group consisting of 2-amino-1-propanol, 1-amino-2-propanol, 3-amino-1-propanol, 2-amino-propane-1,3-diol, 1-amino-2-butanol, 4-amino-2-butanol, 4-amino-1-butanol, 3-amino-1-butanol, 3-amino-2-butanol, 2-amino-1-butanol, dimethyl glucamine, 2,5-diaminocyclohexane-1,4-diol, 2-aminocycohexan-1-ol and 2,4,6-triaminocyclohexane-1,3,5-triol,
b) ≥0.7 wt. % to ≤8.0 wt. %, more preferably ≥0.7 wt. % to ≤6.0 wt. %, hydrogen peroxide,
c) at least one primary dye precursor are selected from the group consisting of resorcinol, 2-methylresorcinol, 4-chlororesorcinol, 2-chlororesorcinol, m-aminophenol, hydroxybenzomorpholine, 2,4-diaminophenoxyethanol, 4-amino-2-hydroxytoluene, 2-methyl-5-hydroxyethylaminophenol, 5- ((2-hydroxyethyl)amino)-1,3-benzodioxol and salts thereof,
d) at least one oxidative coupler dye precursor selected from the group consisting of resorcinol, 2-methylresorcinol, 4-chlororesorcinol, 2-chlororesorcinol, m-aminophenol, hydroxybenzomorpholine, 2,4-diaminophenoxyethanol, 4-amino-2-hydroxytoluene, 2-methyl-5-hydroxyethylaminophenol, 5-((2-hydroxyethyl)amino)-1,3-benzodioxol and salts thereof and
e) at least one buffering agent

In one embodiment, the presently claimed invention is directed to a hair colouring composition which is a two-component hair colouring comprising a tint composition (A) and an oxidizing composition (B); wherein the tint composition (A) comprises
a) at least one alkalizing agent of general formula (I) as described herein,
c) at least one oxidative primary dye precursor as described herein; and
d) at least one oxidative coupler dye precursor as described herein
and
wherein the oxidizing composition (B) comprises
b) at least one oxidizing agent as described herein and
e) at least one buffering agent as described herein.

In one embodiment of the presently claimed invention, a method of treating hair with the composition therefore preferably comprises the steps of:
a. providing a tint composition as described herein;
b. providing an oxidizing composition as described herein;
c. mixing the oxidizing composition and the tint composition to obtain a mixed hair colouring composition as described herein;
d. applying the mixed hair colouring composition for the oxidative dyeing of keratin fibers onto the hair;
e. leaving the composition on the hair for 2 to 50 minutes; and
f. subsequently rinsing the composition from the hair.

In one embodiment of the presently claimed invention, the hair colouring composition is preferably obtained by mixing immediately prior to use a tint composition and the oxidizing composition. A sufficient amount of the mixed colouring composition is applied to the hair, according to the hair abundance, generally from 20 to 250 grams depending on the amount of hair to be coloured. Upon such preparation, the colouring composition is applied to the hair to be dyed and remains in contact with the hair for an amount of time effective to dye the hair. Typically, the hair colouring composition is allowed to act on the hair from 2 to 50, preferably 3 to 40, preferably 3 to 25 minutes, at a temperature ranging from 15 to 50° C. Thereafter, the hair is rinsed with water to remove the composition and dried. If necessary, the hair is washed with a shampoo and rinsed, e.g., with water or a weakly acidic solution, such as a citric acid or tartaric acid solution, and dried. Optionally, a separate conditioning product may also be provided.

The hair colouring composition can be applied on hair via an applicator bottle or brush. It can be used on full head or partly on single strands (highlight application) as common highlight applicator foils, caps and special applicators can be used, but also freehand techniques such as balayage, with brush and/or combs can be possible. The composition can also be applied as a mousse via a manual spray, a pressurized container or an aerosol mousse. The composition may be dispensed as a solid form to which water is added to generate the oxidant and form a thickened vehicle suitable for hair colouring.

The invention is associated with one or more of the following advantages:
none to low lift of the hair
a wider range of desired colour shades for colouring the hair
low levels of damage of the hair
a pleasant odour profile while application to the hair
reduced levels of dye adducts
less off-tone

### EXAMPLES

The following examples illustrate the formulations and performance results according to the presently claimed invention. These examples are not intended to be inclusive of all aspects of the subject matter disclosed herein, but rather to illustrate representative compositions and results. These examples are not intended to exclude equivalents and variations of the presently claimed invention, which are apparent to one skilled in the art.

### Method of Measurements

### 1. Hair Damage Measurement via FT-IR

Damage caused to the hair was assessed by a FT-IR (Fourier Transform Infrared) method, which was established to be suitable for studying the effects of keratin surface damage. Strassburger, J., J. Soc. Cosmet Chem., 36, 61-74 (1985); Joy, M. & Lewis, D. M., Int. J. Cosmet. Sci., 13, 249-261 (1991); Signori, V. and Lewis, D. M., Int. J. Cosmet. Sci., 19, 1-13 (1997)). In particular, these authors have shown that the method was suitable for quantifying the amount of cysteic acid. In general, the oxidation of cystine is thought to be a suitable marker by which to monitor the overall oxidation of the keratinous part of the fiber. Net, the measurement of cysteic acid units by FT-IR was commonly used.

Signori and Lewis (D. M., Int. J. Cosmet. Sci., 19, 1-13 (1997)) showed that FT-IR using a diamond Attenuated Total Internal Reflection (ATR) cell was a sensitive and reproducible way of measuring the cysteic acid content of single fibers and bundles. Hence, the method that was employed to measure the cysteic acid content of multiple fibre bundles and full hair switches, was based upon the FTIR diamond cell ATR method employed by Signori and Lewis (1997). The detailed description of the method for testing the different damage inhibitors follows thereafter:

A Perkin Elmer Spectrum® 1 Fourier Transform Infrared (FTIR) composition equipped with a diamond Attenuated Total Internal Reflection (ATR) cell was used to measure the cysteic acid concentration in mammalian or synthetic hair. In this method, hair switches of various sizes and colours were used. The switches were platted (˜1 plait per cm) in order to minimize variations in surface area of contact between readings. The Oxidative hair Treatment Protocol described above was repeated for 5 cycles to mimic the behaviour of hair after repeated bleaching cycles. Following this treatment, four readings per switch were taken (⅓ and ⅔s down the switch on both sides), and an average calculated. Backgrounds were collected every 4 readings, and an ATR cell pressure of 1 N/m was employed. The cell was cleaned with ethanol between each reading, and a contamination check was performed using the monitor ratio mode of the instrument. As prescribed by Signori &amp; Lewis in 1997, a normalized double derivative analysis routine was used. The original spectra was initially converted to absorbance, before being normalized to the 1450 cm⁻¹ band (the characteristic and invariant protein CH₂ stretch). This normalized absorbance was then twice derivatised using a 13-point averaging. The value of the 1450 cm⁻¹ normalized 2nd derivative of the absorbance at 1040 cm⁻¹ was taken as the relative concentration of cysteic acid. This figure was multiplied by −1×10⁴ to recast it into suitable units.

### 2. Colour Measurement via Spectrophotometer

A Minolta spectrophotometer CM-2600d was used to measure the colour of the cured and dried hair tresses, five points on both the front and back sides, and the average D65 L*a*b values reported.

### 3. Adduct Determination via HPLC Measurement

A HPLC system Ultimate 300Q RSCL (Thermo Fisher), including Ultimate 3000 RS pump (high pressure gradient pump) and Ultimate 3000 RS Diode Array detector is used within the analysis. HPLC separations were performed on a reversed phase C18 HPLC column (e.g. Kinetex EvoC18 Core Shell, 150×3.0 mm, 2.6 μm, Phenomenex). All solvents are HPLC-MS grade. HPLC gradient is used with a flow of 0.4 ml/min, 10 ul injection volume, with the column at 20° C. (+/−2° C.). The gradient conditions used were as follows:

| Time (minutes): | 25 mM NH₄ HCOO | MeOH |
|---|---|---|
| 0 | 85 | 15 |
| 12 | 65 | 35 |
| 24 | 10 | 90 |
| 27 | 10 | 90 |
| 30 | 85 | 15 |
| 33 | 85 | 15 |

Detector signals recorded: UV-VIS (200-800 nm) and MS (m/z=50-1000 amus, ESI in positive mode). Compound identity was confirmed by MS and UV-VIS, quantification was typically done at 230-800 nm.

### Tested Alkali Materials

A series of alkali materials were tested to determine which gave the best overall performance. Table 1 shows two alkali materials known within the area of hair colourants, and 2-amino-1-propanol, 2-amino-1-butanol and dimethyl glucamine (DMG) as the new inventive alkali material.

TABLE 1
List of tested alkali materials

| Compound | Structure | Comment |
|---|---|---|
| Ammonia | NH₃ | not within the scope of the presently |
| | | claimed invention |
| Monoethanolamine | | not within the scope of the presently |
| (MEA) | | claimed invention |
| 2-amino-1-propanol | | |
| (2A1P) | | |
| 2-amino-1-butanol (2A1B) | 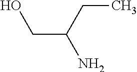 | |
| Dimethyl glucamine | 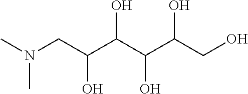 | |

### Assessment of Overall Performance of Alkalizing Agents Comparing to known Alkalizing Agents

The overall performance of the new alkalizing agents according to the general formula (I) was assessed versus the requirements of;
1. Being able to deliver low to no unpleasant odour (example 1),
2. Being able to reduce the amount of undesired adduct formation (example 2)
3. Being able to deliver a broader dye shade palette consumers desire by lowering dye adduct levels versus monoethanolamine (MEA) (example 3).

### Example 1: Comparing Alkali “Odour” Performance

A series of tint compositions were created with the same molar level of the alkali under investigation, as shown in Table 2a. The same molar level was used to ensure a rigorous comparison between the materiais. Table 2a shows the tint composition formulations in terms of weight percent of the different materials used.

Table 2b shows the oxidative composition in terms of weight percentage of the materials used. Table 2b ingredients were mixed with the tint composition given in Table 2a and provided ready to use axidative hair colouring formulas.

TABLE 2a
Tint compositions with different alkalizing agents

| | Composition | Composition | Composition | Composition |
|---|---|---|---|---|
| | 1A* | 1B* | 1C | 1D |
| Tint Ingredients | (% w/w) | (% w/w) | (% w/w) | (% w/w) |
| DI water | q.s | q.s | q.s | q.s |
| Disodium EDTA (BASF) | 0.100 | 0.100 | 0.100 | 0.100 |
| Ascorbic acid (OSKAR BERG) | 0.300 | 0.300 | 0.300 | 0.300 |
| Sodium Sulfite (BCD CHEMIE) | 0.400 | 0.400 | 0.400 | 0.400 |
| Ammonia Solution (25%) | 4.527 | | | |
| (Brenntag GmbH) | | | | |
| Monoethanolamine (MEA) | | 4.066 | | |
| (SASSOL) | | | | |
| 2-amino-1-propanol (2A1P) | | | 5.000 | |
| (Angus) | | | | |
| 2-amino-1-butanol (2A1B) | | | | 5.933 |
| (Angus) | | | | |
| Crème pre-mix formula | 40.000 | 40.000 | 40.000 | 40.000 |
| Cetearyl Alcohol (BASF) | (10.000) | (10.000) | (10.000) | (10.000) |
| Steareth-20 (CRODA) | (3.000) | (3.000) | (3.000) | (3.000) |
| DI Water | (27.000) | (27.000) | (27.000) | (27.000) |

q.s: quantity sufficient
*not within the scope of the presently claimed invention

TABLE 2b
Oxidative composition (1E) (3% Developer)

| Ingredients | Amount (% w/w) |
|---|---|
| Water Purified (q.s) | q.s. |
| Salicylic Acid | 0.10 |
| Disodium Phosphate | 2.00 |
| Phosphoric Acid (85%) | 3.00 |
| Etidronic Acid | 0.01 |
| Hydrogen peroxide (50% Hydroxide | 6 |
| peroxide Interox co-SO, SOLVAY) | |
| Water Purified (q.s) | q.s. |
| Crème pre-mix formula | 20.00 |
| Cetearyl Alcohol (BASF) | (5.00) |
| Steareth-20 (CRODA) | (1.50) |
| DI Water | (13.50) |

q.s: quantity sufficient

The formulations were prepared according to the following methods.

Crème pre-mix formula was a part of either the tint formulation or the oxidizing formulation. A crème pre-mix is created by combining together DI water (68.5%), cetearyl alcohol (25%) and Steareth-20 (6.5%). The mixture was heated to 80-85° C. to melt the waxes and surfactant with stirring. The composition was homogenized for 5 minutes and then cooling was started to reduce the product to room temperature. The resulting crème pre-mix was thick and used in the batches 1A-1D and also within the oxidizing composition.

### Tint Compositions:

The remaining components of the tint formula for each product were mixed together at room temperate with DI water. When all of the materials were dissolved, 40 wt. % of the crème pre-mix was added, and the mixture stirred until a uniform consistency was obtained. The products were then stored in aluminium tubes until used.

### Oxidizing Compositions:

The remaining components were mixed together with the DI water at room temperature. When all of the materials were dissolved, 20 wt. % of the crème pre-mix was added, and the mixture stirred until a uniform consistency was obtained. The products were then stored in plastic bottles until used.

In the examples of the presently claimed invention either 2% or 3% oxidizing formula was used.

### Sniff Testing Method for Comparing Alkali “Odour” Performance

10 g of tint compositions of 1A to 1D were mixed with 10 g of an oxidizing composition (Welloxon Perfect 6% Developer from the Wella Company) (tint and oxidizing compositions are mixed 1:1 in weight ratios) within a hair colouring bowl.

The sniff test showed that the MEA, 2A1P and 2A1B provided the low odour profile that consumers desire. Ammonia showed a distinctive odour which was not preferred.

### Example 2: Adduct Formation

TABLE 3
Tint compositions with different alkali media.

| | Composition | Composition | Composition | Composition |
|---|---|---|---|---|
| | 2A* | 2B* | 2C | 2D |
| Tint Ingredients | (% w/w) | (% w/w) | (% w/w) | (% w/w) |
| DI water | q.s | q.s | q.s | q.s |
| Disodium EDTA | 0.100 | 0.100 | 0.100 | 0.100 |
| Ascorbic acid | 0.300 | 0.300 | 0.300 | 0.300 |
| Sodium Sulfite | 0.400 | 0.400 | 0.400 | 0.400 |
| 2-methoxymethyl-p- | 0.951 | 0.951 | 0.951 | 0.951 |
| phenylenediamine Primary Dye | | | | |
| (DRAGON CHEMICALS) | | | | |
| Resorcinol | 0.688 | 0.688 | 0.688 | 0.688 |
| Coupler Dye (ACETO) | | | | |
| Ammonia Solution (25%) | 4.527 | | | |
| Monoethanolamine (MEA) | | 4.066 | | |
| 2-amino-1-propanol (2A1P) | | | 5.000 | |
| 2-amino-1-butanol (2A1B) | | | | 5.933 |
| Crème pre-mix | 40.000 | 40.000 | 40.000 | 40.000 |
| (Cetearyl Alcohol) | (10.000) | (10.000) | (10.000) | (10.000) |
| (Steareth-20) | (3.000) | (3.000) | (3.000) | (3.000) |
| (DI Water) | (27.000) | (27.000) | (27.000) | (27.000) |

*not within the scope of the presently claimed invention

The tint compositions were prepared in a similar way to the compositions used in example 1. The components were mixed together until fully dissolved, and then the crème pre-mix was added and the product mixed until being homogenous.

The tint compositions were created with the same molar level of alkalizing agents. The same molar level was used to ensure a consistent comparison between the materials. The prepared tint compositions were then mixed with the oxidizing composition (1E) as described above.

### Method of Applying Hair Colouring Formulation to Hair

All products and equipment for the hair dye-out were equilibrated to 30° C. in a circulation oven overnight. Level 5 hair tresses (Kerling International Haarfabrik GmbH, Backnang, Germany) were preselected by weight. Tresses weighing in total 900 mg (+/−10 mg) were found to have 630 mg of free hair, accessible to the mixed creme colouring composition in the following dye-out process.

Equal weights (approximately 4 g each) of tint composition (2A-2D) and oxidizing composition (1E) were mixed in a bowl using a dye-out brush, pH was adjusted with phosphoric acid to ensure pH of the mixture between 6.8 and 7.2. This mixture was referred to as the mixed colouring composition as claimed in the presently claimed invention.

Approximately 1260 mg (+/−20 mg) of colouring composition was applied to the free hair portion of a tress (weight ratio colouring composition: hair ˜2:1) and the colour developed at 30° C. for 30 minutes in a 30° C. circulation oven. After colour development and removal from the oven, excess colouring composition is squeezed out from the tress by hand, wearing nitrile gloves.

Table 4 below shows the formation of regular dye products as well as undesired mono and/or di-adduct formation. These regular dyes and adduct formations are than quantified by HPLC-MS method as described above and the result being shared in Table 5 and Table 6 below.

TABLE 4
List of formed regular dyes and dye adducts within examples 2A-2D

| | Species | Structure |
|---|---|---|
| (Formed in Ammonia System—2A tint composition) | Regular Dye Product (and its isomers) | 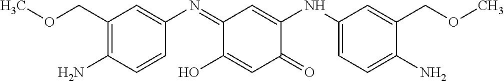 |
| (Formed in | Regular | Same as ammonia system |
| MEA System— | | |
| 2B tint | | |
| composition) | | |
| | Mono-Adduct (and its isomers) | 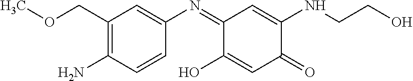 [SMILES: COCc1cc(/N=C2/C=C(NCCO)C(=O)C=C2O)ccc1N] |
| (Formed in | Regular | Same as ammonia system |
| 2A1P | | |
| System—2C | | |
| tint | | |
| composition) | | |
| | Mono-Adduct (and its isomers) | 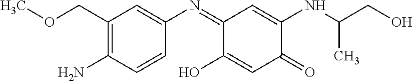 [SMILES: COCc1cc(/N=C2/C=C(NC(C)CO)C(=O)C=C2O)ccc1N] |
| (Formed in | Regular | Same as ammonia system |
| 2A1B | | |
| System—2D | | |
| tint | | |
| composition) | | |
| | Mono-Adduct (and its isomers) | 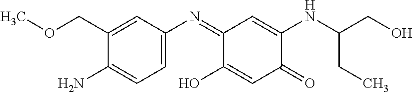 [SMILES: CCC(CO)NC1=C/C(=N/c2ccc(N)c(COC)c2)C(O)=CC1=O] |

TABLE 5
Relative amount of regular dye product in the examples
of using tint compositions 2A-2D with an oxidizing
composition to make the colouring composition

| | Tint composition used in | Regular dye |
|---|---|---|
| Example | colouring composition | product |
| 2a* | 2A (Ammonia) | 29.9% |
| 2b* | 2B (Monoethanolamine) | 42.3% |
| 2c | 2C (2-amino-1-propanol) | 75.2% |
| 2d | 2D (2-amino-1-butanol) | 100% |

*not within the scope of the presently claimed invention

The amounts in colouring composition are given as percentage of the total regular dye product formed for a given alkalizer normalized to the amount of crème recovered from the tresses and normalized to the system resulting in the highest amount of regular dye product.

The amount of formed regular dye product clearly shows that under low pH conditions the ammonia system (example 2a), as well as the monoethanolamine system (example 2b) produced only little regular dye product in comparison to 2-amino-1propanol (example 2c) and 2-amino-1-butanol (example 2d). Surprisingly, example 2d showed 3.3 times higher regular dye product formation in comparison to example 2a and 2.4 times higher regular dye product formation compared to the example 2b. The same trend was observed for 2 C as well.

TABLE 6
Relative adduct formation amount in the examples
of using tints compositions 2A-2D with an oxidizing
composition to make the colouring composition

| | Tint composition used in | Regular dye | Mono-adduct |
|---|---|---|---|
| Example | colouring composition | product | formation |
| 2e* | 2A (Ammonia) | 100% | 0% |
| 2f* | 2B (Monoethanolamine) | 69.8% | 30.2% |
| 2g | 2C (2-amino-1-propanol) | 92.2% | 7.8% |
| 2h | 2D (2-amino-1-butanol) | 96.0% | 4.0% |

*not within the scope of the presently claimed invention

The amounts of the mono-adduct formation are relative amounts in the colouring composition as percentage of the total regular dye product and mono-adduct formed for a given alkalizer normalized to the amount of crème recovered from the tresses. The ammonia system (example 2e) produced no adducts. The use of 2-amino-1-propanol (example 2g) and 2-amino-1-butanol (example 2h) produced far lower levels of the mono-adduct in the mixed hair colouring composition compared to monoethanolamine (example 2f) The reduced level of mono-adduct formation allows for the use of higher levels of dyes in the shading process.

### Example 3: Dye Shades Enabled by Use of Alkalizing Agents (According to Formula (I)) Compared to MEA

Table 7a and 7b shows tint formulations comprising different alkalizing agents that were used to obtain a broader range of shades.

The tint compositions (3A-3H) were mixed with a 2% hydrogen peroxide oxidizing composition shown in Table 7c (31) and applied to hair to provide a dark brown colour. The pH of the mixed hair colouring formulation was between 6.8 and 7.5 for all mixtures.

The first two examples 3A and 3B produced levels of dye adducts that could not be release for consumer usage. . . . However, when the same dye formulations were transformed into the inventive examples 3C to 3H, the formulations formed the desired dye shades and the level of dye adducts was sufficiently low to enable their use by consumers. As a result, the dye shades could not be formulated with MEA (examples 3A and 3B) and could be formulated using 2A1P, 2A1B and DMG formulations (examples 3C to 3H).

TABLE 7a
Comparison of Different Dye Shade Formation
by using MEA and other alkalizers

| | Ex. 3A* | Ex. 3B* | Ex. 3C | Ex 3D |
|---|---|---|---|---|
| Tint Ingredients | (% w/w) | (% w/w) | (% w/w) | (% w/w) |
| DI water | q.s | q.s | q.s | q.s |
| Disodium EDTA | 0.100 | 0.100 | 0.100 | 0.100 |
| Ascorbic acid | 0.300 | 0.300 | 0.300 | 0.300 |
| Sodium Sulfite | 0.400 | 0.400 | 0.400 | 0.400 |
| Citric acid | 0.200 | 0.100 | 0.200 | 0.100 |

Primary Dyes

| | | | | |
|---|---|---|---|---|
| 2,5-toluenediamine sulfate | 0.100 | | 0.100 | |
| 1,4-Diamino-2-(methoxymethyl)- | | 0.250 | | 0.250 |
| benzene | | | | |
| p-Aminophenol | | 0.800 | | 0.800 |
| 1-Hexyl 4,5-Diamino Pyrazole Sulfate | 1.680 | | 1.680 | |
| m-Aminophenol | 0.650 | 0.160 | 0.650 | 0.160 |

Coupler Dyes

| | | | | |
|---|---|---|---|---|
| 2,4-diaminophenoxyethanol 2HCl | | 0.440 | | 0.440 |
| 2-Methyl-5-Hydroxyethylaminophenol | | 0.800 | | 0.800 |
| 1-Naphthol | | 0.140 | | 0.140 |
| 5-((2-hydroxyethyl)amino)-1,3- | 0.500 | | 0.500 | |
| benzodioxol | | | | |

Alkalizers

| | | | | |
|---|---|---|---|---|
| Monoethanolamine* | 1.300 | 0.600 | | |
| 2-amino-1-propanol | | | 1.600 | 0.735 |
| Crème pre-mix | 40.000 | 40.000 | 40.000 | 40.000 |
| Cetearyl Alcohol | (10.000) | (10.000) | (10.000) | (10.000) |
| Steareth-20 | (3.000) | (3.000) | (3.000) | (3.000) |
| DI Water | (27.000) | (27.000) | (27.000) | (27.000) |

*not within the scope of the presently claimed invention

TABLE 7b
Comparison of Different Dye Shade Formation
by using MEA and other alkalizers

| | Ex. 3E | Ex. 3F | Ex. 3G | Ex. 3H |
|---|---|---|---|---|
| Tint Ingredients | (% w/w) | (% w/w) | (% w/w) | (% w/w) |
| DI water | q.s | q.s | q.s | q.s |
| Disodium EDTA | 0.100 | 0.100 | 0.100 | 0.100 |
| Ascorbic acid | 0.300 | 0.300 | 0.300 | 0.300 |
| Sodium Sulfite | 0.400 | 0.400 | 0.400 | 0.400 |
| Citric acid | 0.200 | 0.100 | 0.200 | 0.100 |

Primary Dyes

| | | | | |
|---|---|---|---|---|
| 2,5-toluenediamine sulfate | 0.100 | | 0.100 | |
| 1,4-Diamino-2-(methoxymethyl)- | | 0.250 | | 0.250 |
| benzene | | | | |
| p-Aminophenol | | 0.800 | | 0.800 |
| 1-Hexyl 4,5-Diamino Pyrazole Sulfate | 1.680 | | 1.680 | |
| m-Aminophenol | 0.650 | 0.160 | 0.650 | 0.160 |

Coupler Dyes

| | | | | |
|---|---|---|---|---|
| 2,4-diaminophenoxyethanol 2HCl | | 0.440 | | 0.440 |
| 2-Methyl-5-Hydroxyethylaminophenol | | 0.800 | | 0.800 |
| 1-Naphthol | | 0.140 | | 0.140 |
| 5-((2-hydroxyethyl)amino)-1,3- | 0.500 | | 0.500 | |
| benzodioxol | | | | |

Alkalizers

| | | | | |
|---|---|---|---|---|
| 2-amino-1-butanol | 1.900 | 0.875 | | |
| Dimethyl glucamine | | | 8.840 | 4.080 |
| Crème pre-mix | 40.000 | 40.000 | 40.000 | 40.000 |
| Cetearyl Alcohol | (10.000) | (10.000) | (10.000) | (10.000) |
| Steareth-20 | (3.000) | (3.000) | (3.000) | (3.000) |
| DI Water | (27.000) | (27.000) | (27.000) | (27.000) |

TABLE 7c
Oxidative composition (3I) (2% Developer)

| Ingredients | Amount (% w/w) |
|---|---|
| Water Purified (q.s) | q.s. |
| Salicylic Acid | 0.10 |
| Disodium Phosphate | 0.30 |
| Phosphoric Acid (85%) | 0.18 |
| Etidronic Acid | 0.01 |
| Hydrogen peroxide (50% Hydroxide | 4 |
| peroxide Interox co-SO, SOLVAY) | |
| Water Purified (q.s) | q.s. |
| Crème pre-mix formula | 20.00 |
| Cetearyl Alcohol (BASF) | (5.00) |
| Steareth-20 (CRODA) | (1.50) |
| DI Water | (13.50) |

q.s: quantity sufficient

The components of the oxidative compositions with the exception of phosphoric Acid (85%) were mixed together with the DI water at room temperature. When all of the materials were dissolved, 20 wt. % of the creme pre-mix was added, and the mixture stirred until a uniform consistency was obtained. After addition of phosphoric Acid (85%) the pH was checked to be between 2.3 and 2.6. The products were filled and stored in plastic bottles until used.

### Example 4: Hair Colouring Formulations With Different Levels of 2-amino-1-propanol and Different Levels of Buffering Agents

Table 8 summarizes a series of tint compositions that are identical to tint composition 1C, but contain varying levels of 2-amino-1-propanol. The tint compositions were prepared by using the of example 1. These tint compositions were mixed in a ratio of 1:1 with oxidizing compositions that are identical to composition 1E, but contain varying levels of buffering agents (disodium phosphate and phosphoric acid) equivalent to identify optimum concentrations of alkalizing agents. In examples 4N and 4P disodium phosphate and phosphoric acid were completely removed and replaced with another buffering agent (citric acid).

The level of alkalizing agent (2-amino-1-propanol) within the tint compositions was varying between 0.1 wt. % and 8.0 wt. % in the tint composition prior to mixing with an oxidizing composition. After mixing the tint composition with the oxidizing composition, the level of alkalizing agent ranges from 0.05 wt. % to 4.0 wt. %

The pH of the compositions is determined by using either a Mettler Toledo MP220 or a MP225 pH equipment, fitted with a standard laboratory pH electrode. The equipment is calibrated prior to the use by using standard calibration buffers and using standard calibration procedure.

The tint composition 1C given in Table 2a with the oxidizing composition 1E given in Table 2b was used in the hair colouring mixtures given in Table 8 within different buffering agents.

According to Table 8, the results of the level study with 2-amino-1-propanol in connection with different buffering agents according to the presently claimed invention show that the desired pH below 9 was reached with concentrations of 2-amino-1-propanol in tint composition between 0.1% and 8%, which later mixed 1:1 with oxidant composition.

TABLE 8
Hair Colouring Formulations with Different Levels of Buffering Agents (in oxidizing
composition, 1E) and Different Levels of 2A1P (in tint composition, 1C)

| | | | | | | | | | | | | | | | |
|---|---|---|---|---|---|---|---|---|---|---|---|---|---|---|---|
| Examples (w/w %) | 4A | 4B | 4C | 4D | 4E | 4F | 4G | 4H | 4I | 4J | 4K | 4L | 4M | 4N | 4P |

Oxidizing composition (1E) with different buffering agent

| | | | | | | | | | | | | | | | |
|---|---|---|---|---|---|---|---|---|---|---|---|---|---|---|---|
| Disodium Phosphate | 3.00 | 3.00 | 3.00 | 3.00 | 3.00 | 2.00 | 2.00 | 2.00 | 0.20 | 0.20 | 0.20 | 0.15 | 0.15 | | |
| Phosphoric acid | 4.00 | 4.00 | 4.00 | 4.00 | 4.00 | 3.00 | 3.00 | 3.00 | 0.20 | 0.20 | 0.20 | 0.15 | 0.15 | | |
| Citric acid | | | | | | | | | | | | | | 0.10 | 0.10 |

Tint composition (1C) with different levels of 2A1P

| | | | | | | | | | | | | | | | |
|---|---|---|---|---|---|---|---|---|---|---|---|---|---|---|---|
| 2A1P | 5.00 | 5.90 | 6.90 | 7.25 | 8.00 | 4.00 | 4.50 | 5.00 | 0.10 | 0.30 | 0.50 | 0.10 | 0.30 | 5.90 | 8.00 |
| pH | 6.94 | 7.38 | 7.75 | 8.89 | 8.96 | 6.43 | 6.89 | 8.24 | 6.61 | 7.14 | 7.64 | 8.44 | 8.51 | 7.03 | 8.41 |

### Example 5: Hair Colouring Formulations of Mixed Alkali Systems

In table 9, examples of mixed alkali systems are shown which use more than one alkali agent to provide the desired level of buffering to the hair colouring composition. The tint compositions 5A to 5E were prepared according to the method described above.

TABLE 9
Example tint compositions showing the impact of mixed alkali systems.

| | Ex. 5A* | Ex. 5B* | Ex. 5C | Ex. 5D | Ex. 5E |
|---|---|---|---|---|---|
| Tint Ingredient | (% w/w) | (% w/w) | (% w/w) | (% w/w) | (% w/w) |
| DI water | q.s | q.s | q.s | q.s | q.s |
| Disodium EDTA | 0.100 | 0.100 | 0.100 | 0.100 | 0.100 |
| Ascorbic acid | 0.300 | 0.300 | 0.300 | 0.300 | 0.300 |
| Sodium Sulfite | 0.400 | 0.400 | 0.400 | 0.400 | 0.400 |

Primary Dyes

| | | | | | |
|---|---|---|---|---|---|
| 2-methoxymethyl-p-phenylenediamine | 0.250 | 0.250 | 0.250 | 0.250 | 0.250 |
| p-Aminophenol | 0.800 | 0.800 | 0.800 | 0.800 | 0.800 |
| 4,5-diamino-1-hexylpyrazol sulfate | 0.050 | 0.500 | 0.500 | 0.500 | 0.500 |
| N,N-Bis(2-hydroxyethyl)-p- | 0.050 | 0.050 | 0.050 | 0.050 | 0.050 |
| phenylenediamine | | | | | |

Coupler Dyes

| | | | | | |
|---|---|---|---|---|---|
| 2-methylresorcinol | 0.292 | 0.292 | 0.292 | 0.292 | 0.292 |
| 4-Amino-2-hydroxytoluene | 0.208 | 0.208 | 0.208 | 0.208 | 0.208 |
| m-Aminophenol | 0.670 | 0.608 | 0.608 | 0.608 | 0.608 |

Alkalisers

| | | | | | |
|---|---|---|---|---|---|
| Ammonia Solution (25%) | 4.527 | | | 2.264 | |
| Monoethanolamine | | 4.066 | | | 2.033 |
| 2-amino-1-propanol | | | 5.000 | 2.500 | 2.500 |
| Crème pre-mix | 40.000 | 40.000 | 40.000 | 40.000 | 40.000 |
| (Cetearyl Alcohol) | (10.000) | (10.000) | (10.000) | (10.000) | (10.000) |
| (Steareth-20) | (3.000) | (3.000) | (3.000) | (3.000) | (3.000) |
| (DI Water) | (27.000) | (27.000) | (27.000) | (27.000) | (27.000) |

*not within the scope of the presently claimed invention

The tint compositions are mixed in a ratio of 1:1 with the oxidizing composition (1E with 3% developer).

The mixed alkali colouring compositions with tint compositions 5D and 5E and oxidizing compositions show lower levels of adducts versus the single alkali compositions (5A and 5B). For the mixture of ammonia and 2-amino-1-propanol, the level of 2A1P dye adducts is about a half. . . . The combination with ammonia has an additional advantage in that the product has a reduced odour profile compared to the ammonia alone product. For the mixture of MEA and 2-amino-1-propanol (5E), the level of MEA dye adduct is reduced by around a third, and the level of 2-amino-1-propanol based dye adduct is reduced by about a half. The mixed systems are able to further reduce the adduct levels, even though the molar level of the total alkali is kept constant.

1. An oxidative hair colouring composition comprising
a) at least one alkalizing agent of the general formula (I), 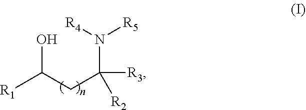
wherein
R1 is hydrogen or linear or branched C1-C6 alkyl which is unsubstituted or substituted with 1, 2, 3 or 4 OH,
R2 is hydrogen or linear or branched C1-C6 alkyl which is unsubstituted or substituted with 1 or 2 OH,
R3 is hydrogen or linear or branched, unsubstituted C2-C6 alkyl,
or
1 R2 and R3 together with the carbon atoms to which they are bonded form a saturated 5-, 6-, 7-or 8-membered carbocyclic ring that is unsubstituted or substituted with 1, 2, 3, or 4 substituents selected from the group consisting of OH and NH₂,
R4 is hydrogen or linear or branched, unsubstituted C1-C6 alkyl,
R5 is hydrogen or linear or branched, unsubstituted C1-C6 alkyl, and
n is 0, 1, 2 or 3;
whereby the compound of general formula (I) is not monoethanolamine;
b) at least one oxidizing agent;
c) at least one oxidative primary dye precursor;
d) at least one oxidative coupler dye precursor and
e) at least one buffering agent.

2. (canceled)

3. (canceled)

4. (canceled)

5. The oxidative hair colouring composition according to claim 1, wherein the component a) is present in an amount in the range of ≥0.05 wt. % to ≤5.0 wt. %, based on the total weight of the oxidative hair colouring composition.

6. (canceled)

7. (canceled)

8. (canceled)

9. (canceled)

10. The oxidative hair colouring composition according to claim 1, wherein the component b) is present in an amount in the range of ≥0.7 wt. % to ≤8.0 wt. %, based on the total weight of the oxidative hair colouring composition.

11. (canceled)

12. (canceled)

13. (canceled)

14. (canceled)

15. (canceled)

16. The oxidative hair colouring composition according to claim 1, wherein the component e) is selected from buffering alkali compounds and buffering acidic compounds.

17. (canceled)

18. (canceled)

19. (canceled)

20. (canceled)

21. (canceled)

22. The oxidative hair colouring composition according to claim 1, wherein the component e) is present in an amount in the range of ≥0.05 wt. % to ≤4.0 wt. %, based on the total weight of the oxidative hair colouring composition.

23. The oxidative hair colouring composition according to claim 1 comprising,
a) ≥0.05 wt. % to ≤5.0 wt. % of alkalizing agents selected from the group consisting of 2-amino-1-propanol, 1-amino-2-propanol, 3-amino-1-propanol, 2-amino-propane-1,3-diol, 1-amino-2-butanol, 4-amino-2-butanol, 4-amino-1-butanol, 3-amino-1-butanol, 3-amino-2-butanol, 2-amino-1-butanol, dimethyl glucamine, 2,5-diaminocyclohexane-1,4-diol, 2-aminocycohexan-1-ol and 2,4,6-triaminocyclohexane-1,3,5-triol,
b) ≥0.7 wt. % to ≤8.0 wt. % hydrogen peroxide,
c) at least one primary dye precursor,
d) at least one oxidative coupler dye precursor and
e) at least one buffering agent
wherein the wt. % are each based on the total weight of the oxidative hair colouring composition.

24. The oxidative hair colouring composition according to claim 1 comprising,
a) ≥0.1 wt. % to ≤4.0 wt. % of alkalizing agents selected from the group consisting of 2-amino-1-propanol, 1-amino-2-propanol, 2-amino-1-butanol and dimethyl glucamine,
b) ≥0.7 wt. % to ≤8.0 wt. % hydrogen peroxide,
c) at least one primary dye precursor,
d) at least one oxidative coupler dye precursor and
e) at least one buffering agent
wherein the wt. % are each based on the total weight of the oxidative hair colouring composition.

25. (canceled)

26. The oxidative hair colouring composition according to claim 1, wherein the oxidative hair colouring composition has a pH in the range from ≥6.5 to ≤8.5.

27. The oxidative hair colouring composition according to claim 1, wherein the composition further comprises at least one direct dye selected from the group consisting of Acid Yellow 1, Disperse Red 17, Basic Brown 17, Acid Black 1, Picramic acid, HC Red 13, N,N′-Bis(2-hydroxyethyl)-2-nitro-p-phenylenediamine, HC Blue 2, HC Yellow 4, HC Yellow 2, HC Orange 1, HC Red 1, HC Red 3, 4-Amino-3-nitrophenol, 3-Nitro-p-hydroxyethylaminophenol, 4-Nitrophenyl aminoethylurea, HC Red 10, HC Red 11, 2-Hydroxyethyl picramic acid, HC Blue 12, Hydroxyethyl-2-nitro-p-toluidine, HC Yellow 12, HC Yellow 7, 2-Chloro-6-(ethylamino)-4-nitrophenol, 2-Amino-6-Chloro-4-Nitrophenol, 4-Hydroxypropylamino-3-nitrophenol, HC Yellow 13, 2,6-Diamino-3- ((pyridin-3-yl)azo)pyridine, Basic violet 2, Basic Red 51, HC Blue 16, Basic Orange 1, Basic Red 76, Basic Brown 16, Basic Yellow 57, CI14700, Acid Orange 7, FD&C Yellow 6, Acid Red 33, Acid Yellow 23, Acid Blue 9, Acid violet 9, Acid Red 92, Acid Yellow 3, 1-Amino-4-hydroxy-9,10-anthracendion, CI 60725, Acid violet 43, Disperse Violet 1, Carbon black, Disperse Black 9, Curry red, 2-Hydroxy-1,4-naphthoquinone, Lawsonia Inermis Cera, Indigofera tinctoria, HC Blue 14, CI 42053, Acid Red 52, Acid Green 25, Disperse Blue 377, Pigment Red 57, HC Blue 15, Tetrabromphenol Blue, Anthocyanins, Chlorophyllin-Copper complex, Annatto, Natural Green 3, Betanin, Capsanthin, Basic Yellow 29 and combinations thereof.

28. The oxidative hair colouring composition according to claim 1, wherein the composition comprises at least one chelant selected from the group consisting of ethylenediamine tetraacetic acid (EDTA), ethylenediamine-N,N′-disuccinic acid (EDDS), ethylenediamine-N,N′-diglutaric acid (EDDG), 2-hydroxypropylenediamine-N,N′-disuccinic acid (HPDDS), glycinamide-N,N′-disuccinic acid (GADS), ethylenediamine-N-N′-bis(ortho-hydroxyphenyl acetic acid) (EDDHA), dimethly glucamine (DMG), salts, derivatives, and mixtures thereof.

29. (canceled)

30. The oxidative hair colouring composition according to claim 1, wherein the composition comprises at least one fatty substance selected from the group consisting of linear or branched, optionally cyclic, C6-C16 alkanes, non-silicone oils of animal, plant, mineral or synthetic origin, fatty alcohols, fatty acids, fatty acid esters and/or fatty alcohol esters, non-silicone waxes, silicones and mixtures thereof.

31. (canceled)

32. (Canceled

33. The oxidative hair colouring composition according to claim 1, wherein the composition comprises at least one thickener selected from the group consisting of polymeric thickeners, mineral thickeners and organic thickeners and gel network thickener systems and/or combinations thereof.

34. (canceled)

35. The oxidative hair colouring composition according to claim 1, wherein the composition comprises at least one non-ionic surfactant.

36. (canceled)

37. (canceled)

38. The oxidative hair colouring composition according to claim 1, wherein the hair colouring formulation comprises a gel network thickener system formed from at least one surfactant and at least one fatty alcohol.

39. (canceled)

40. (canceled)

41. (canceled)

42. (canceled)

43. (canceled)

44. (canceled)

45. The oxidative hair colouring composition according to claim 1, wherein the composition comprises a second alkalizing agent selected from the group consisting of ammonia and monoethanolamine.

46. The oxidative hair colouring composition according to claim 15, wherein the molar ratio of ammonia to component a) is in the range of 5.0:1.0 to 1.0:5.0.

47. The oxidative hair colouring composition according to claim 15, wherein the molar ratio of monoethanolamine to component a) is in the range of 5.0:1.0 to 1.0:5.0.

48. A kit comprising:
A) a tint composition comprising components a), c), d) and optionally e) as defined in claim 1 and
B) an oxidative composition comprising component b) and optionally e) as defined in claim 1.

49. A method of treating hair with the composition according to claim 1 comprises the steps of:
a. providing a tint composition (A) comprising components a), c), d) and optionally e) as defined in claim 1,
b. providing an oxidative composition (B) comprising component b) and optionally e) as defined in claim 1,
c. mixing the tint composition (A) and the oxidative composition (B) to obtain a mixed hair colouring composition according to claim 1,
d. applying the hair colouring composition onto the hair;
e. leaving the composition on the hair for a period in the range from ≥2 to ≤50 minutes; and
f. subsequently rinsing the composition from the hair.

50. The method for treating hair according to claim 49, wherein the tint composition (A) is mixed with the oxidative composition (B) in a weight ratio in the range of 3.0:1.0 to 1.0:3.5.

51. (canceled)

52. (canceled)
